(19) United States
(12) Patent Application Publication
(10) Pub. No.: US 2023/0274851 A1
(43) Pub. Date: Aug. 31, 2023

(54) SURFACE ELECTRODE

(71) Applicant: Murata Manufacturing Co., Ltd., Nagaokakyo-shi (JP)

(72) Inventors: Tomoki OUCHI, Nagaokakyo-shi (JP); Yoichi MORIYA, Nagaokakyo-shi (JP); Kahori TAKATSUKI, Nagaokakyo-shi (JP); Koji TANAKA, Nagaokakyo-shi (JP)

(21) Appl. No.: 18/173,123

(22) Filed: Feb. 23, 2023

(30) Foreign Application Priority Data
Feb. 25, 2022 (JP) 2022-028412
Dec. 13, 2022 (JP) 2022-198818

(51) Int. Cl. H01B 1/04; H01B 5/14; A61B 5/25

(52) U.S. Cl. CPC H01B 1/04; H01B 5/14; A61B 5/25; A61B 2562/0217; A61B 2562/04; A61B 2562/164; A61B 2562/168

(57) ABSTRACT

A surface electrode having a first surface and a plurality of electrode elements disposed on the first surface and spaced from each other in a manner so as to be configured to contact a measured surface of an object to be measured; a stretchable wire electrically connecting the plurality of electrode elements; and a stretchable insulator covering a side of the stretchable wire adjacent to the electrode elements.

### CROSS REFERENCE TO RELATED APPLICATIONS

The present application claims priority to Japanese Patent Application No. 2022-028412, filed Feb. 25, 2022, and Japanese Patent Application No. 2022-198818, filed Dec. 13, 2022, the entire contents of each of which are incorporated herein by reference.

### BACKGROUND OF THE INVENTION

### Field of the Invention

The present invention relates to a surface electrode.

### Description of the Related Art

In recent years, there has been demand for electrodes used to measure changing surfaces, such as biological skins and device surfaces. For biomedical measurement, for example, the application of such electrodes is not limited to electrocardiogram and electroencephalograph. The application of biomedical electrodes for acquiring biological signals during exercise has been expanding.

As electrodes used on changing surfaces, for example, metal electrodes and wires have been used without making any modifications thereto (see, e.g., Japanese Unexamined Patent Application Publication No. 2012-146900).

### SUMMARY OF THE INVENTION

When an electrode is subjected to tensile or contraction stress from a changing surface, it has been difficult for the electrode to follow the surface changes. It has therefore been difficult to avoid the occurrence of noise.

An object of the present invention is to provide an electrode that is capable of following surface changes and causes less noise.

A surface electrode according to the present invention has a first surface and a plurality of electrode elements disposed on the first surface and spaced from each other in a manner so as to be configured to contact a measured surface of an object to be measured; a stretchable wire electrically connecting the plurality of electrode elements; and a stretchable insulator covering a side of the stretchable wire adjacent to the plurality of electrode elements.

The surface electrode according to the present invention includes the wire and the insulator that are both stretchable. When used as a biomedical electrode, the surface electrode can follow changes of a body surface and causes less noise.

### BRIEF DESCRIPTION OF THE DRAWINGS

FIG. 1A is a schematic perspective view illustrating a configuration of a surface electrode according to Embodiment 1;

FIG. 1B is a schematic cross-sectional view illustrating a cross-sectional structure of an area A indicated by a closed dotted line in FIG. 1A;

FIG. 1C is a transparent plan view illustrating a planar arrangement of components of the surface electrode illustrated in FIG. 1A;

FIG. 2A is a schematic cross-sectional view illustrating the position of the surface electrode where an expansion and contraction direction in which stress is applied is upward in the Z axis direction, and a wire is subjected to the stress on a side;

FIG. 2B is a schematic diagram illustrating an example in which stress is applied upward to the side of the wire of the surface electrode illustrated in FIG. 2A;

FIG. 2C is a schematic diagram illustrating an example in which stress is applied downward to a curve of the wire of the surface electrode illustrated in FIG. 2A;

FIG. 3A is a schematic cross-sectional view illustrating a cross-sectional shape of a wire 4 illustrated in FIG. 2A;

FIG. 3B is a schematic cross-sectional view illustrating a cross-sectional shape of another wire 4a;

FIG. 3C is a schematic cross-sectional view illustrating a cross-sectional shape of another wire 4b;

FIG. 3D is a schematic cross-sectional view illustrating a cross-sectional shape of another wire 4c;

FIG. 3E is a schematic cross-sectional view illustrating a cross-sectional shape of another wire 4d;

FIG. 3F is a schematic cross-sectional view illustrating a cross-sectional shape of another wire 4e;

FIG. 3G is a schematic cross-sectional view illustrating a cross-sectional shape of another wire 4f;

FIG. 3H is a schematic cross-sectional view illustrating a cross-sectional shape of another wire 4g;

FIG. 3I is a schematic cross-sectional view illustrating a cross-sectional shape of another wire 4h;

FIG. 4A is a schematic cross-sectional view illustrating the position of a surface electrode according to Embodiment 2 where an expansion and contraction direction in which stress is applied is downward in the Z axis direction, and the wire is subjected to the stress on a side;

FIG. 4B is a schematic diagram illustrating an example in which stress is applied upward to the side of the wire of the surface electrode illustrated in FIG. 4A;

FIG. 4C is a schematic diagram illustrating an example in which stress is applied downward to the curve of the wire of the surface electrode illustrated in FIG. 4A;

FIG. 5A is a schematic cross-sectional view of a surface electrode according to Embodiment 3 in which the electrode elements and the wire are electrically connected, with a via interposed therebetween, and the surface electrode is subjected to tensile force in the in-plane direction;

FIG. 5B is a schematic cross-sectional view of an example in which the wire connecting to the via on the side thereof is subjected to tensile force in the in-plane direction;

FIG. 5C is a schematic cross-sectional view illustrating deformation of the wire and the via subjected to the tensile force in the in-plane direction illustrated in FIG. 5B;

FIG. 6 is a schematic diagram illustrating an example where a stress applied in the in-plane direction in FIG. 5B is decomposed into components, a force vertical to the plane and a force horizontal to the plane;

FIG. 7A is a schematic cross-sectional view of a surface electrode according to Embodiment 4 in which the electrode elements and the wire are electrically connected, with the via interposed therebetween, and the surface electrode is subjected to tensile force in the in-plane direction;

FIG. 7B is a schematic cross-sectional view of an example in which the wire connecting to the via on the curve thereof is subjected to tensile force in the in-plane direction;

FIG. 7C is a schematic cross-sectional view illustrating deformation of the wire and the via subjected to the tensile force in the in-plane direction illustrated in FIG. 7B;

FIG. 8 is a schematic diagram illustrating an example where a stress applied in the in-plane direction in FIG. 7B is decomposed into components, a force vertical to the plane and a force horizontal to the plane;

FIG. 9 is a schematic cross-sectional view illustrating a cross-sectional structure of a surface electrode according to Embodiment 5;

FIG. 10 is a schematic cross-sectional view illustrating a cross-sectional structure of a surface electrode according to Embodiment 6;

FIG. 11 is a schematic cross-sectional view illustrating a cross-sectional structure of an insulator covering an electrode element illustrated in FIG. 10;

FIG. 12 is a schematic cross-sectional view illustrating a cross-sectional structure of a surface electrode according to Embodiment 8;

FIG. 13A is a schematic cross-sectional view illustrating a cross-sectional structure of a surface electrode according to Embodiment 9;

FIG. 13B is a schematic cross-sectional view illustrating an example in which there are seams, each extending along the joint between the electrode element and the insulator to reach the wire;

FIG. 14A to FIG. 14E are schematic bottom views illustrating various patterns of a planar arrangement of the wire;

FIG. 15 is a schematic cross-sectional view illustrating a cross-sectional structure of insulators and the wire in a surface electrode according to Embodiment 10;

FIG. 16A to FIG. 16C are schematic cross-sectional views illustrating a process of manufacturing the cross-sectional structure of the insulators and the wire illustrated in FIG. 15;

FIG. 17 is a schematic cross-sectional view illustrating a cross-sectional structure of the insulators and the wire in a surface electrode according to Embodiment 11;

FIG. 18A to FIG. 18C are schematic cross-sectional views illustrating a process of manufacturing the cross-sectional structure of the insulators and the wire illustrated in FIG. 17;

FIG. 19 is a schematic cross-sectional view illustrating a cross-sectional structure of first and second insulators and the wire in a surface electrode according to Embodiment 12, as viewed in a direction perpendicular to the longitudinal direction of the wire 4;

FIG. 20 is a schematic cross-sectional view illustrating a cross-sectional structure of the insulators and the wire in a surface electrode according to Embodiment 13;

FIGS. 21A to 21D are schematic cross-sectional views illustrating a process of manufacturing the cross-sectional structure of the insulators and the wire illustrated in FIG. 20;

FIG. 22 is a schematic bottom view illustrating an arrangement of the electrode elements and the insulator in a surface electrode according to Embodiment 14;

FIG. 23 is a schematic cross-sectional view illustrating a cross-sectional structure of a surface electrode according to Embodiment 15;

FIG. 24 is a schematic cross-sectional view illustrating a cross-sectional structure of a surface electrode according to Embodiment 16;

FIG. 25 is a schematic cross-sectional view illustrating a cross-sectional structure of a surface electrode according to Embodiment 17;

FIG. 26 is a schematic cross-sectional view illustrating a cross-sectional structure of a surface electrode according to another example of Embodiment 17;

FIG. 27 is a schematic bottom view illustrating an arrangement of the electrode elements and the insulator in a surface electrode according to Embodiment 18;

FIG. 28 is a schematic cross-sectional view illustrating a cross-sectional structure of a surface electrode according to Embodiment 19;

FIG. 29 is a schematic cross-sectional view illustrating a cross-sectional structure of a surface electrode according to another example of Embodiment 19;

FIG. 30A to FIG. 30F are schematic bottom views illustrating various patterns of a planar arrangement of the wire 4;

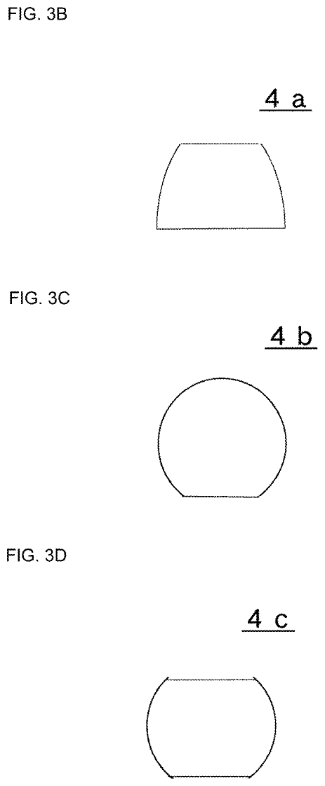

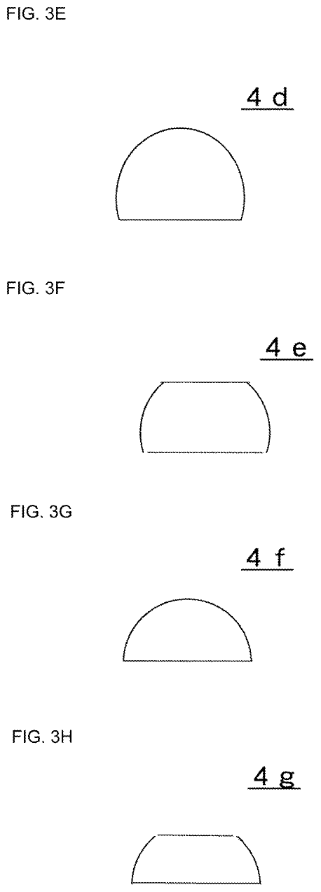

FIG. 3IA to FIG. 3IE are schematic bottom views illustrating various patterns of a planar arrangement of the wire 4 where a distance between adjacent ones of the electrode elements in an expansion and contraction direction differs from that in a direction perpendicular to the expansion and contraction direction;

FIG. 32A is a schematic diagram illustrating an example in which the surface electrode according to Embodiment 1 is attached to a knee of a leg, and FIG. 32B is a schematic diagram illustrating a bent position of the knee illustrated in FIG. 32A;

FIG. 33A is a plan view of a surface electrode including a wire for a test;

FIG. 33B is a schematic cross-sectional view illustrating a cross-sectional structure of the surface electrode, as viewed in the direction F-F of FIG. 33A;

FIG. 34A is a plan view of the same surface electrode as that in FIG. 33A;

FIG. 34B is a plan view illustrating the surface electrode of FIG. 34A deformed by tensile force applied thereto in the X direction;

FIG. 35A is a schematic cross-sectional view illustrating a cross-sectional structure of a surface electrode according to Embodiment 20;

FIG. 35B is an enlarged cross-sectional view of a region G indicated in FIG. 35A by a dotted line, which encloses one electrode element at an end portion of the surface electrode;

FIG. 35C is an enlarged cross-sectional view illustrating a separation at the end portion of the surface electrode illustrated in FIG. 35B;

FIG. 35D is an enlarged cross-sectional view illustrating a separation in a surface electrode of a reference example which does not include a sealing portion for sealing the wire;

FIG. 36 is a schematic cross-sectional view illustrating a cross-sectional structure of a surface electrode according to Embodiment 21;

FIG. 37A is a schematic cross-sectional view illustrating a cross-sectional structure of a surface electrode according to Embodiment 22;

FIG. 37B is an enlarged cross-sectional view of a region H indicated in FIG. 37A by a dotted line, which encloses one electrode element at an end portion of the surface electrode;

FIG. 37C is an enlarged cross-sectional view illustrating a separation at the end portion of the surface electrode illustrated in FIG. 37B;

FIG. 37D is an enlarged cross-sectional view illustrating a separation in a surface electrode of a reference example which does not include a sealing portion for sealing the wire;

FIG. 38A is a schematic cross-sectional view illustrating a cross-sectional structure of a surface electrode according to Embodiment 23;

FIG. 38B is an enlarged cross-sectional view of a region I indicated in FIG. 38A by a dotted line, which encloses one electrode element at an end portion of the surface electrode, the enlarged cross-sectional view illustrating a crack formed at the end portion of the surface electrode;

FIG. 38C is an enlarged cross-sectional view illustrating a crack in a surface electrode of a reference example which does not include a sealing portion for sealing the wire;

FIG. 39A is a schematic cross-sectional view illustrating a cross-sectional structure of a surface electrode according to Embodiment 24;

FIG. 39B is an enlarged cross-sectional view of a region J indicated in FIG. 39A by a dotted line, which encloses one electrode element at an end portion of the surface electrode;

FIG. 39C is an enlarged cross-sectional view illustrating a crack in a surface electrode of a reference example which does not include a sealing portion serving as a protective layer covering an outer side portion of the insulator;

FIG. 40A is a schematic cross-sectional view illustrating a cross-sectional structure of a surface electrode according to Embodiment 25; and

FIG. 40B is an enlarged cross-sectional view of a region K indicated in FIG. 40A by a dotted line, which encloses one electrode element at an end portion of the surface electrode.

### DETAILED DESCRIPTION OF THE PREFERRED EMBODIMENTS

A surface electrode according to Aspect 1 is a surface electrode having a first surface and a plurality of electrode elements disposed on the first surface and spaced from each other in a manner so as to be configured to contact a measured surface of an object to be measured; a stretchable wire electrically connecting the plurality of electrode elements; and a stretchable insulator covering a side of the stretchable wire adjacent to the plurality of electrode elements.

According to Aspect 2, in the surface electrode of Aspect 1, in a plan view of a cross section orthogonal to the first surface, the stretchable wire may have a contour containing a straight line parallel to the first surface and a curve.

In this configuration, the stretchable insulator may be shaped to conform to the contour of the stretchable wire at the interface between the stretchable insulator and the stretchable wire.

According to Aspect 3, in the surface electrode of Aspect 2, the surface electrode may further have a second surface opposite the first surface, and a distance between the straight line and the first surface may be shorter than a distance between the straight line and the second surface.

According to Aspect 4, in the surface electrode of Aspect 2, the surface electrode may further have a second surface opposite the first surface, and a distance between the straight line and the first surface may be longer than a distance between the straight line and the second surface.

According to Aspect 5, in the surface electrode of any one of Aspects 2 to 4, a total length of the curve may be longer than a total length of the straight line.

According to Aspect 6, in the surface electrode of any one of Aspects 2 to 5, the insulator may cover the plurality of electrode elements, with the plurality of electrode elements at least partially exposed at the first surface.

According to Aspect 7, in the surface electrode of any one of Aspects 2 to 6, the stretchable insulator may have a recess between adjacent ones of the plurality of electrode elements.

According to Aspect 8, in the surface electrode of any one of Aspects 2 to 7, in a plan view of a cross section orthogonal to the first surface, the stretchable insulator between the plurality of electrode elements may be in contact with an entire perimeter of the stretchable wire, as viewed in a direction orthogonal to the cross section.

According to Aspect 9, in the surface electrode of any one of Aspects 2 to 8, the stretchable wire may contain gallium.

According to Aspect 10, in the surface electrode of any one of Aspects 2 to 9, the stretchable insulator may include a first layer and a second layer, and in a plan view of a cross section orthogonal to the first surface, the first layer and the second layer may be disposed with the stretchable wire sandwiched therebetween, as viewed in a direction orthogonal to the cross section.

According to Aspect 11, in the surface electrode of any one of Aspects 2 to 9, the stretchable insulator may include a first layer, a second layer, and a third layer, and in plan view of a cross section orthogonal to the first surface, the first layer, the second layer, the stretchable wire, and the third layer may be disposed in the described order, with the first layer being closest to the first surface, as viewed in a direction orthogonal to the cross section.

According to Aspect 12, in the surface electrode of any one of Aspects 1 to 11, in a plan view of the first surface viewed in a direction orthogonal to the first surface, an area occupied by the plurality of electrode elements may be greater than an area outside the plurality of electrode elements.

According to Aspect 13, in the surface electrode of any one of Aspects 1 to 12, a shortest distance between two adjacent ones of the plurality of electrode elements may be greater than a thickness of any one of the plurality of electrode elements.

According to Aspect 14, in the surface electrode of any one of Aspects 1 to 13, a material of the stretchable wire may be more stretchable than that of the plurality of electrode elements.

According to Aspect 15, in the surface electrode of any one of Aspects 1 to 14, the surface electrode may further have a second surface opposite the first surface and may further include a substrate on the second surface, and the substrate may be made of a material that is softer than that of the electrode elements.

According to Aspect 16, in the surface electrode of Aspect 15, the material of the substrate may be harder than that of the stretchable wire.

According to Aspect 17, in the surface electrode of Aspect 15, the material of the substrate may be softer than that of the stretchable wire.

According to Aspect 18, the surface electrode of any one of Aspects 1 to 17 may include a via between the stretchable wire and the plurality of electrode elements, the via electrically connecting the stretchable wire to the plurality of electrode elements, and the via may be made of a mixture of a conductive material and a resin material.

According to Aspect 19, in the surface electrode of Aspect 18, in the mixture of the conductive material and the resin material forming the via, the conductive material may be a carbon-based conductive material.

According to Aspect 20, in the surface electrode of Aspect 18 or 19, in a plan view of a cross section orthogonal to the first surface, the stretchable wire may extend beyond an outside diameter of an end portion of the via toward an end portion of the surface electrode, as viewed in a direction orthogonal to the cross section.

According to Aspect 21, in the surface electrode of any one of Aspects 1 to 20, a distance between the stretchable wire and a second surface opposite the first surface may be shorter than a distance between the stretchable wire and the first surface.

According to Aspect 22, in the surface electrode of any one of Aspects 1 to 22, a length of the stretchable wire in a direction toward the closest electrode element of the plurality of electrode elements on the first surface may be longer than a length of the closest electrode element.

According to Aspect 23, in the surface electrode of any one of Aspects 1 to 22, wherein a first distance between a first set of adjacent electrode elements of the plurality of electrode elements spaced apart in an expansion and contraction direction of the measured surface is longer than a second distance between a second set of adjacent electrode elements of the plurality of electrode elements spaced apart in a direction perpendicular to the expansion and contraction direction.

According to Aspect 24, in the surface electrode of any one of Aspects 1 to 23, the plurality of electrode elements each may have a protrusion protruding in a direction orthogonal to the first surface, and the protrusion may be embedded in the stretchable insulator or in the stretchable wire.

Surface electrodes according to embodiments will now be described with reference to accompanying drawings. Note that substantially the same components in the drawings are denoted by the same reference numerals.

### Embodiment 1

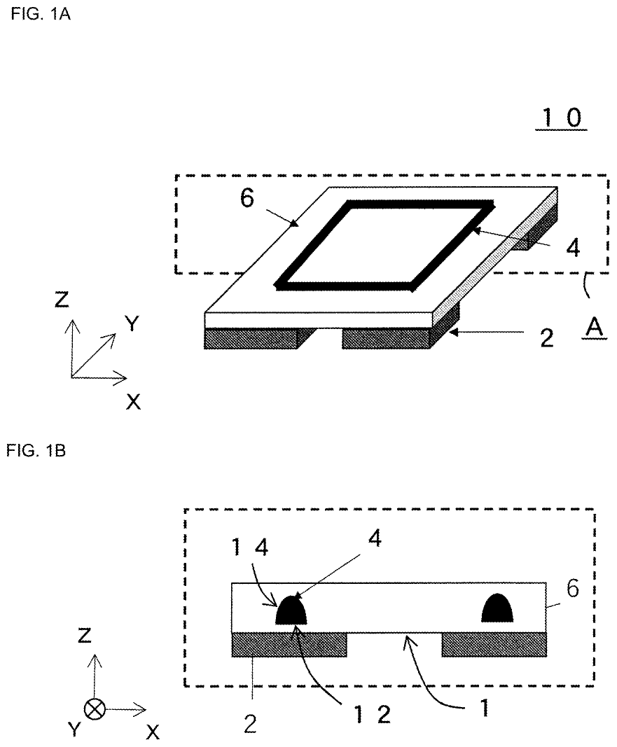

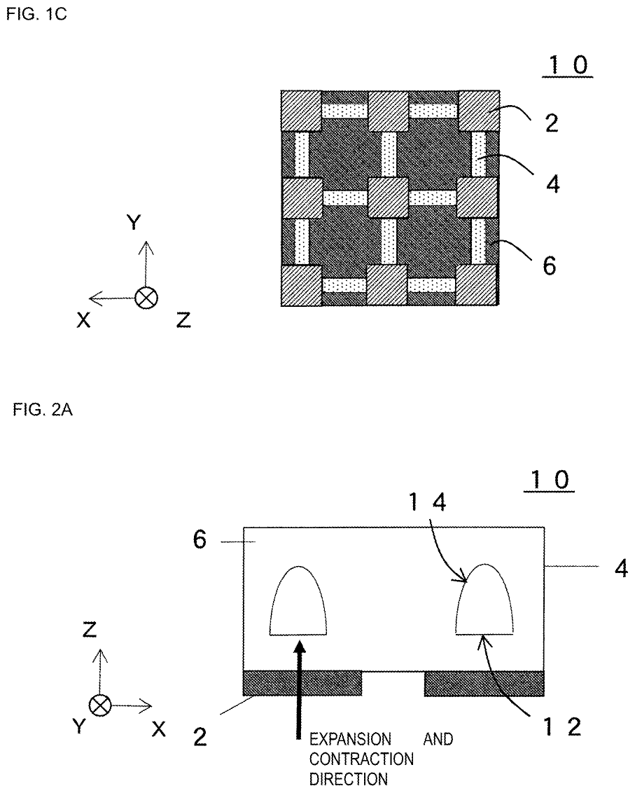

FIG. 1A is a schematic perspective view illustrating a configuration of a surface electrode 10 according to Embodiment 1. FIG. 1B is a schematic cross-sectional view illustrating a cross-sectional structure of an area A indicated by a closed dotted line in FIG. 1A. FIG. 1C is a transparent plan view illustrating a planar arrangement of components of the surface electrode 10 illustrated in FIG. 1A. That is, in FIG. 1C, an insulator 6 is partially seen through when viewed upward from the electrode elements 2. In FIG. 1B and FIG. 1C, for convenience, a plane facing a measured surface of an object to be measured is defined as an XY plane, and a direction perpendicular to the XY plane is defined as a Z direction.

The surface electrode 10 according to Embodiment 1 includes the electrode elements 2 disposed on a first surface 1, a wire 4 having stretchability and configured to electrically connect the electrode elements 2, and the insulator 6 having stretchability and configured to cover a side of the wire 4 adjacent to the electrode elements 2.

In the surface electrode 10 according to Embodiment 1, both the wire 4 and the insulator 6 are stretchable. This allows the surface electrode 10 to follow even such changes as expansion and contraction of the measured surface of the object to be measured. The occurrence of noise can thus be reduced.

Components of this surface electrode will now be described.

Electrode Elements

The electrode elements 2 are spaced from each other and disposed on the first surface 1. The electrode elements 2 are made of a metal, such as copper, silver, gold, or aluminum. The electrode elements 2 may be rectangular in shape, as illustrated in FIG. 1A, FIG. 1B, and FIG. 1C. The shape of the electrode elements 2 is not limited to a rectangle. For example, the electrode elements 2 may be circular, polygonal, or may have a shape containing a straight line and a curve.

Wire

The wire 4 is configured to electrically connect the electrode elements 2 and is stretchable. Being “stretchable” means being elastically deformable. Of various types of deformation caused by applying force to an object, elastic deformation refers to a deformation which allows the object to return to its original shape once the applied force is removed. Therefore, even when the measured surface of the object to be measured changes and the distance between two adjacent ones of the electrode elements 2 changes, the wire 4 can elastically deform to accommodate changes in the distance and respond to movement of the electrode elements 2. The occurrence of noise can thus be reduced.

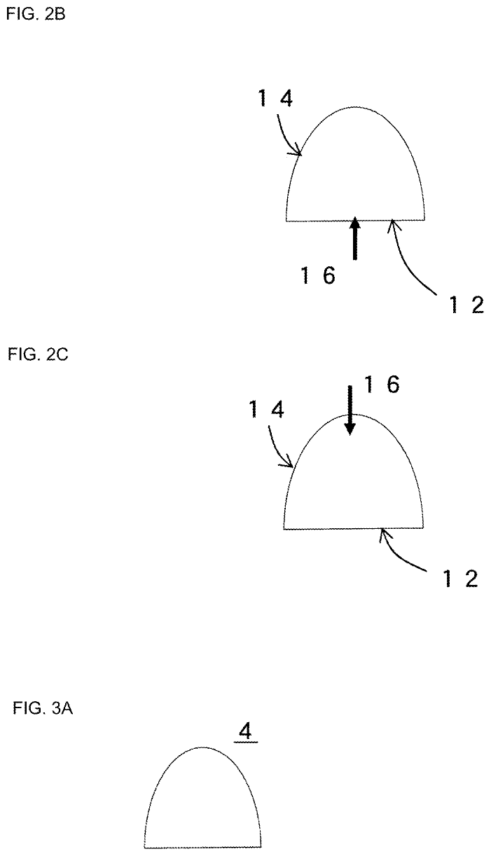

FIG. 2A is a schematic cross-sectional view illustrating the position of the surface electrode 10 where an expansion and contraction direction in which stress is applied is upward in the Z axis direction. FIG. 2B is a schematic diagram illustrating an example in which a stress 16 is applied upward to a side 12 of the wire 4 of the surface electrode 10 illustrated in FIG. 2A. FIG. 2C is a schematic diagram illustrating an example in which the stress 16 is applied downward to a curve 14 of the wire 4 of the surface electrode 10 illustrated in FIG. 2A.

As illustrated in FIG. 2A, the wire 4 has a cross-sectional shape containing one linear side 12 on the lower side in the Z axis direction and the curve 14 on the upper side in the Z axis direction. With this cross-sectional structure having the side 12, the contact at the interface between the wire 4 and a via can be kept stable even during expansion and contraction. The side 12 has a large area of contact with the insulator 6. This enhances electrical contact with the via, improves conductivity, and thus can reduce changes in resistance between the wire 4 and the via.

When the side 12 of the wire 4 is parallel to the measured surface of the object to be measured, downward or upward pressure in the Z axis direction can be relieved. For example, if vibration during exercise of a human (or person), which is an example of the object to be measured, is accompanied by upward stress in the Z axis direction, the side 12 is subjected to the stress as illustrated in FIG. 2B. Since the stress 16 is applied over a large area, the wire 4 is resistant to deformation. This can reduce changes in the cross-sectional area of the wire 4, and can reduce changes in resistance. If downward stress in the Z axis direction is applied, on the other hand, the curve 14 at the top end portion is subjected to the stress 16 as illustrated in FIG. 2C. For example, when downward stress in the Z axis direction is applied, an upward force is also produced as a reaction. Since the insulator and the wire are flexible, however, the upward force is absorbed and what matters here is the source from which the upward force has been produced. Since the area of the top end portion subjected to the stress is smaller than that of the side, the degree of deformation at the top end portion is greater. It is thus necessary, for example, that the occurrence of noise be taken into consideration.

As illustrated in FIG. 1C, the wire 4 may be electrically connected on the upper side of the electrode elements 2 in the Z axis direction and electrically connected to the electrode elements 2, for example, with the side 12 of the wire 4 and the via (not shown) interposed therebetween. The pattern of planar arrangement of the wire 4 is not limited to that illustrated in FIG. 1C.

The wire 4 has a curve on the upper side in the Z axis direction. That is, since the wire 4 has a bulging surface, the number of corners between sides can be reduced. This can reduce the concentration of electric fields, reduce changes in resistance accompanying changes in current path caused by changes in the measured surface of the object to be measured, and reduce the occurrence of noise. When the wire 4 has more curves in cross section, the concentration of electric fields caused by radio frequency radiation can be more effectively relieved, and more noise reduction can be achieved. The concentration of electric fields means that current is concentrated on a particular current path due to radio frequency radiation. Therefore, if the particular current path is closed by deformation, the resulting change in resistance is excessively large. When the concentration of electric fields is relieved to make the current distribution uniform, such a change in resistance can be reduced even if the particular current path is closed by deformation.

If the inner angle between the side and the curve of the wire 4 is an acute angle, the corresponding edge is significantly affected by the skin effect which causes concentration of radio frequency radiation on the surface of the signal line. It is thus preferable that the inner angle between the side and the curve of the wire 4 be an obtuse angle greater than 90°.

FIG. 3A to FIG. 3I are schematic cross-sectional views illustrating cross-sectional shapes of the wire 4 illustrated in FIG. 2A and wires 4a to 4h of other examples. The wire 4 and the wires 4a to 4h have a polygonal shape that contains at least one side and one curve in cross section.

<Insulator>

The insulator 6 is configured to cover a side of the wire 4 adjacent to the electrode elements 2 and is stretchable. Therefore, even when the measured surface of the object to be measured changes and the distance between two adjacent ones of the electrode elements 2 changes, the insulator 6, which is stretchable, can respond to the movement of the electrode elements 2 without reducing elastic deformation of the wire 4. The occurrence of noise can thus be reduced. The insulator 6 may be shaped to conform to the contour of the wire 4 at the interface between the insulator 6 and the wire 4.

The insulator 6 can be made of thermoplastic resin or thermosetting resin commonly used.

### Embodiment 2

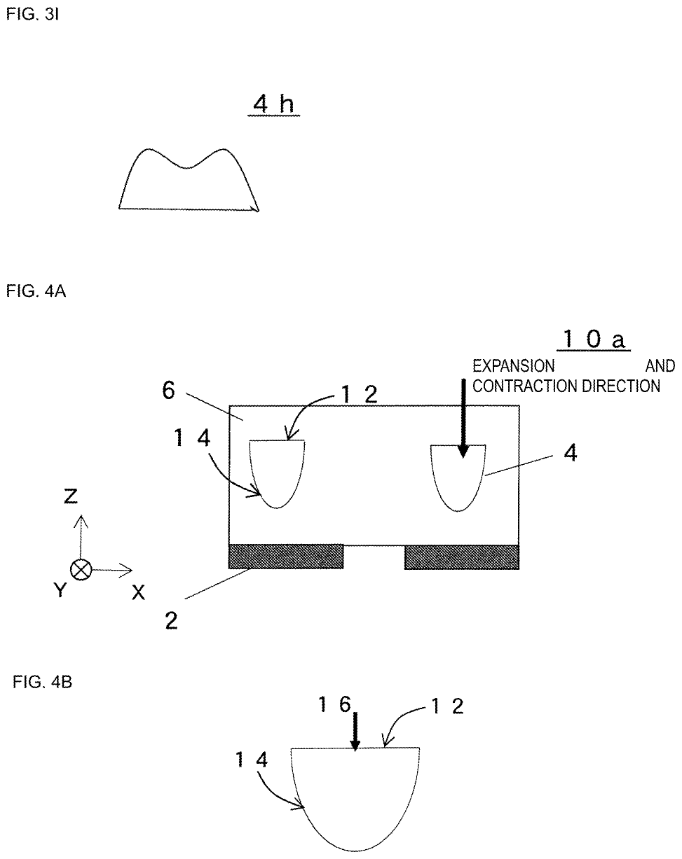

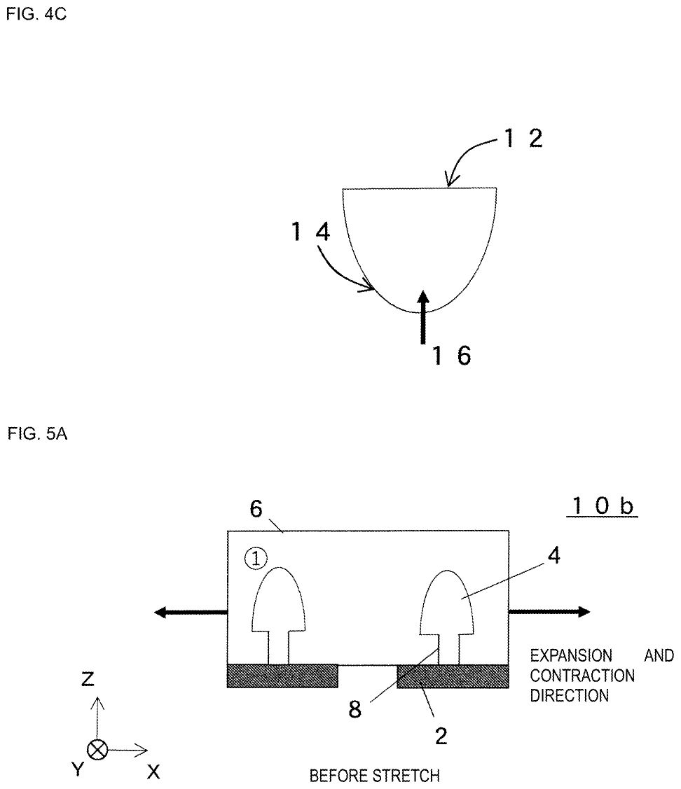

FIG. 4A is a schematic cross-sectional view illustrating the position of a surface electrode 10a according to Embodiment 2 where an expansion and contraction direction in which stress is applied is downward in the Z axis direction, and the wire 4 is subjected to the stress on the side 12. FIG. 4B is a schematic diagram illustrating an example in which the stress 16 is applied downward to the side 12 of the wire 4 of the surface electrode 10a illustrated in FIG. 4A. FIG. 4C is a schematic diagram illustrating an example in which the stress 16 is applied upward in the Z axis direction to the curve of the wire 4 of the surface electrode 10a illustrated in FIG. 4A.

The surface electrode 10a according to Embodiment 2 differs from the surface electrode according to Embodiment 1 in that the wire 4 has the side 12 on the upper side, not on the lower side, in the Z axis direction.

For example, a collision with an external object during exercise of a human (or person), which is an example of the object to be measured, may be accompanied by downward stress from outside the surface electrode 10a in the Z axis direction. In this case, the side 12 is subjected to the stress as illustrated in FIG. 4B. Since the stress 16 is applied over a large area, the wire 4 is resistant to deformation. This can reduce changes in the cross-sectional area of the wire 4, and can reduce changes in resistance. If upward stress in the Z axis direction is applied, on the other hand, the curve 14 at the bottom end portion is subjected to the stress 16 as illustrated in FIG. 4C. In this case, since the area of the bottom end portion subjected to the stress is smaller than that of the side, the degree of deformation at the bottom end portion is greater. It is thus necessary, for example, that the occurrence of noise be taken into consideration.

### Embodiment 3

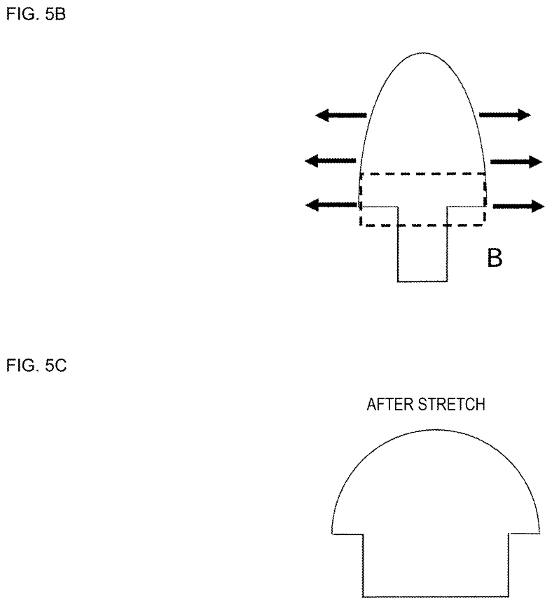

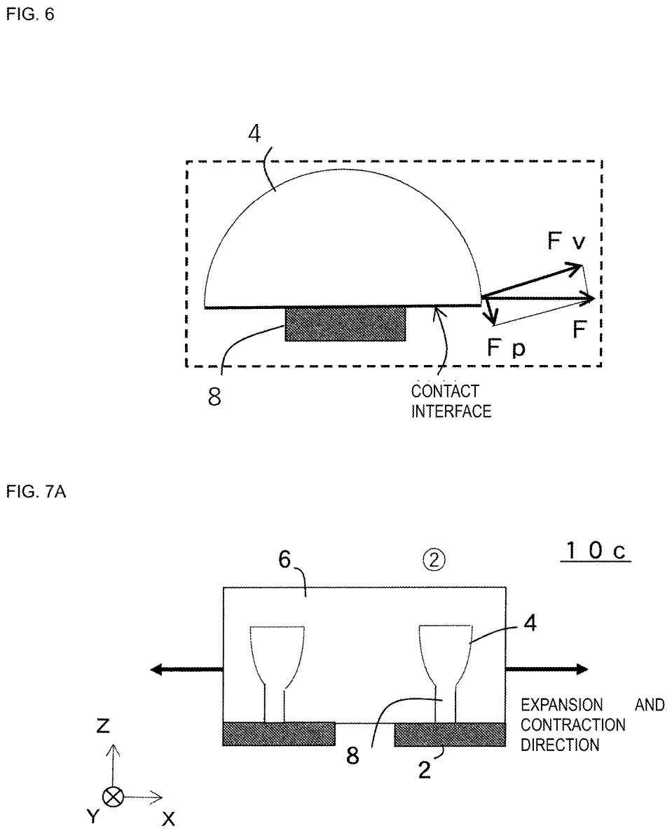

FIG. 5A is a schematic cross-sectional view of a surface electrode 10b according to Embodiment 3 in which the electrode elements 2 and the wire 4 are electrically connected, with a via 8 interposed therebetween, and the surface electrode 10b is subjected to tensile force in the in-plane direction (lateral direction, or XY direction). FIG. 5B is a schematic cross-sectional view of an example in which the wire 4 connecting to the via 8 on the side 12 is subjected to tensile force in the in-plane direction. FIG. 5C is a schematic cross-sectional view illustrating deformation of the wire 4 and the via 8 subjected to the tensile force in the in-plane direction illustrated in FIG. 5B. FIG. 6 is a schematic diagram illustrating an example where a stress F applied in the in-plane direction in FIG. 5B is decomposed into components, a force Fv vertical to the plane and a force Fp horizontal to the plane.

In the surface electrode 10b according to Embodiment 3, the electrode elements 2 and the wire 4 are electrically connected, with the via 8 interposed therebetween. The via 8 passes through the interior of the insulator 6 to connect the electrode elements 2 to the wire 4. That is, the via 8 is insulated from the surrounding by the insulator 6. The via 8 allows electrical connection from the back side of the electrode elements 2, that is, from the side opposite the measured surface. When the wire 4, which is connected to the via 8 on the side 12 as illustrated in FIG. 5B, is subjected to tensile force in the in-plane direction (lateral direction, or XY direction) in this case, a joint portion B of the via 8 is resistant to the force and not easily broken.

Referring to FIG. 6, the force Fp horizontal to the plane is directed downward in the Z axis direction, not in the direction of separation of the contact interface. This indicates that the side 12 of the wire 4 and the via 8 are resistant to separation.

The via 8, which is soft, can absorb stress even when the measured surface changes.

### Embodiment 4

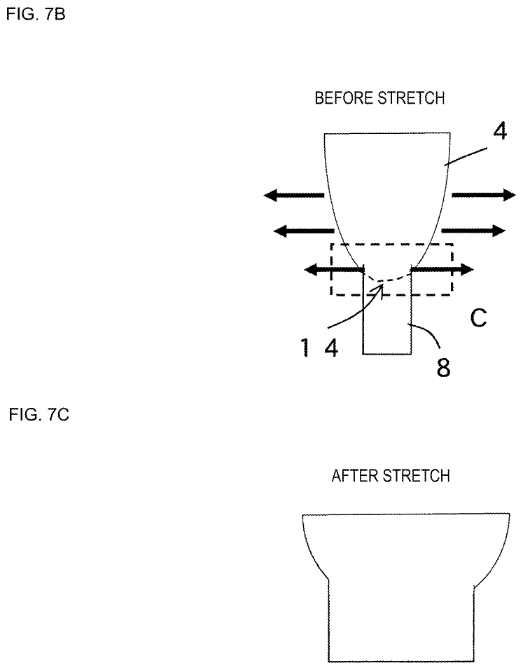

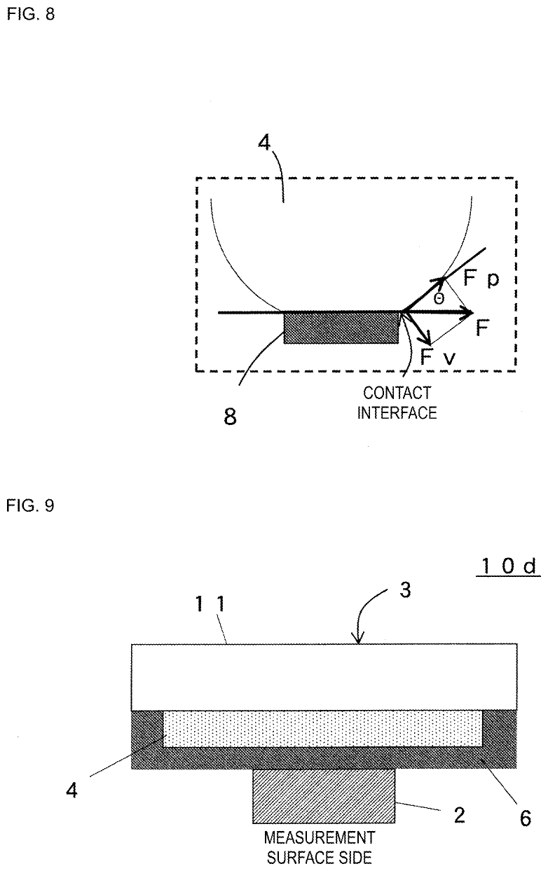

FIG. 7A is a schematic cross-sectional view of a surface electrode 10c according to Embodiment 4 in which the electrode elements 2 and the wire 4 are electrically connected, with the via 8 interposed therebetween, and the surface electrode 10c is subjected to tensile force in the in-plane direction. FIG. 7B is a schematic cross-sectional view of an example in which the wire 4 connecting to the via 8 on the curve 14 is subjected to tensile force in the in-plane direction. FIG. 7C is a schematic cross-sectional view illustrating deformation of the wire 4 and the via 8 subjected to the tensile force in the in-plane direction illustrated in FIG. 7B. FIG. 8 is a schematic diagram illustrating an example where the stress F applied in the in-plane direction in FIG. 7B is decomposed into components, the force Fv vertical to the plane and the force Fp horizontal to the plane.

In the surface electrode 10c according to Embodiment 4, the electrode elements 2 and the wire 4 are electrically connected, with the via 8 interposed therebetween. When the wire 4, which is connected to the via 8 on the curve 14 as illustrated in FIG. 7B, is subjected to tensile force in the in-plane direction (lateral direction, or XY direction) in this case, a joint portion C of the via 8 may be sensitive to the force.

Referring to FIG. 8, the force Fp horizontal to the plane is directed upward in the Z axis direction. This indicates that the force Fp acts in the direction of separation of the curve 14 of the wire 4 from the via 8. Therefore, it would be desirable that the structure described above be used for applications where the surface electrode 10c is not often subjected to force in the in-plane direction.

### Embodiment 5

FIG. 9 is a schematic cross-sectional view illustrating a cross-sectional structure of a surface electrode 10d according to Embodiment 5.

The surface electrode 10d according to Embodiment 5 is characterized in that it includes a substrate 11 having a second surface 3 opposite the measured surface. The substrate 11 and the insulator 6 are configured to cover the wire 4 to bring the components into tight contact. When this ensures airtightness and watertightness, it is possible to prevent oxidation of the wire 4, reduce entry of water toward the wire, and provide greater stability in signal quality.

Substrate

For example, the substrate 11 is made of a material softer than that of the electrode elements 2.

### Embodiment 6

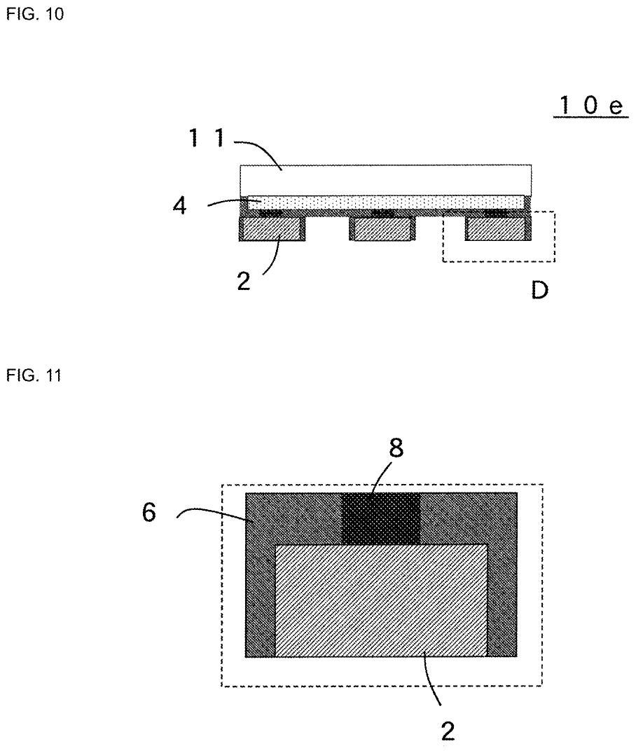

FIG. 10 is a schematic cross-sectional view illustrating a cross-sectional structure of a surface electrode 10e according to Embodiment 6. FIG. 11 is a schematic cross-sectional view illustrating a cross-sectional structure of the insulator 6 covering the electrode element 2 illustrated in FIG. 10.

In the surface electrode 10e according to Embodiment 6, the insulator 6 partially covers the electrode element 2, except the surface for acquiring signals from the measured surface. This can prevent accidental electrical connection between the electrode element 2 and areas outside the measured surface while maintaining electrical connection between the electrode element 2 and the measured surface, and can reduce the occurrence of noise.

### Embodiment 7

In a surface electrode according to Embodiment 7, the wire is made of a material containing gallium. For example, the wire may be made of a material containing 0% to 40% by weight of indium and 60% to 100% by weight of gallium. The material of the wire is not limited to that described above. The wire may be made of EGaIn (with a melting point of 15.5° C.) containing 75.5% by weight of Ga and 24.5% by weight of In, Galinstan (with a melting point of −19° C.) containing 68.5% by weight of Ga, 21.5% by weight of In, and 10% by weight of Sn, or Galinstan (with a melting point of 10° C.) containing 62% by weight of Ga, 25% by weight of In, and 13% by weight of Sn. These materials, which have melting points lower than human body temperature, can keep the wire in liquid form during use of the surface electrode, reduce changes in resistance accompanying expansion and contraction, and suppress noise.

The material of the wire is not limited to the examples described above. For example, the wire may be made of a metal paste containing a resin paste and metal particles dispersed in the resin paste.

### Embodiment 8

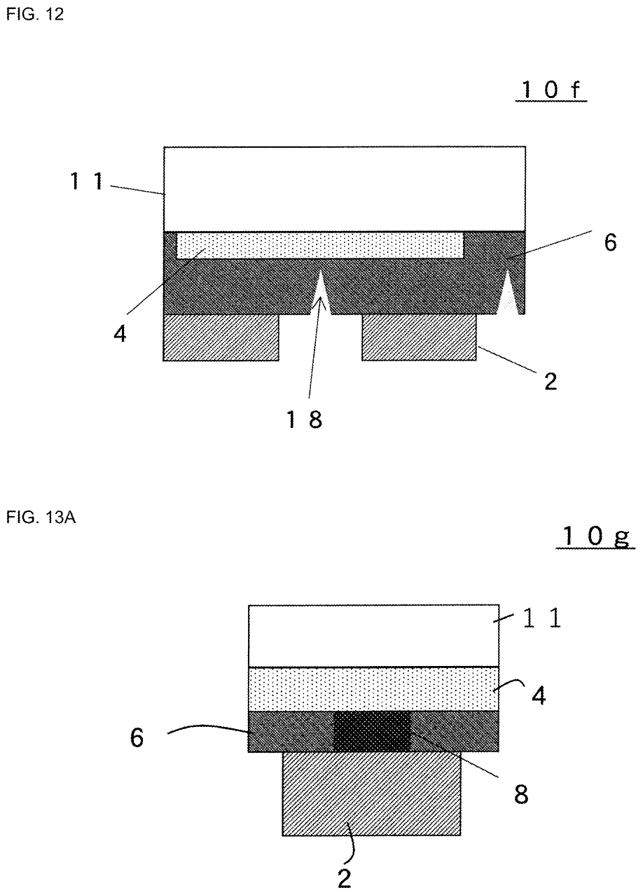

FIG. 12 is a schematic cross-sectional view illustrating a cross-sectional structure of a surface electrode 10f according to Embodiment 8.

In a cross section of the surface electrode 10f according to Embodiment 8 viewed along a plane, the surface electrode 10f has, between adjacent ones of the electrode elements 2, a portion recessed from the measured surface to be in contact with the electrode elements 2. The surface of the recessed portion is covered with the insulator 6. The insulator 6 has notches 18 that are cut in the direction opposite the electrode elements 2.

The structure described above allows air to pass through the notches 18. This allows evaporation of sweat and water collected between the electrode elements 2, and prevents entry of water into the wire 4. There is a high possibility that gaps between the electrode elements 2 and the insulator 6 will serve as a pathway that allows entry of water into the wire 4. The risk of water entry increases as more water collects between the electrode elements 2. The notches 18 improve airflow, make it difficult for water to collect between the electrode elements 2, and thus can reduce entry of water into the wire 4. The notches 18 can improve the performance of following the measured surface.

### Embodiment 9

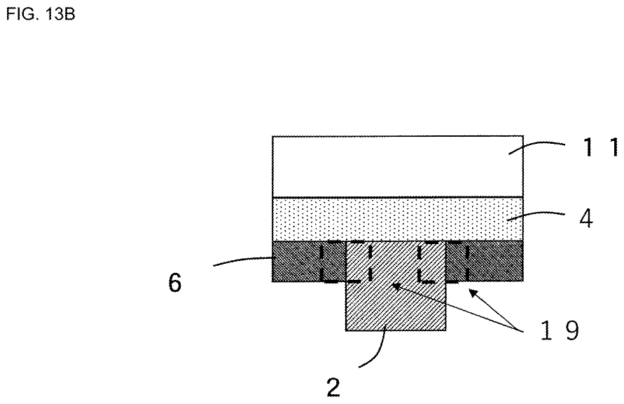

FIG. 13A is a schematic cross-sectional view illustrating a cross-sectional structure of a surface electrode 10g according to Embodiment 9. FIG. 13B is a schematic cross-sectional view illustrating an example in which there are seams 19, each extending along the joint between the electrode element 2 and the insulator 6 to reach the wire 4.

In the surface electrode 10g according to Embodiment 9, the wire 4 is seamlessly covered with the insulator 6 therearound. The term “seam” refers to a gap that extends along the joint between the electrode element 2 and the insulator 6 to reach the wire 4, as described above.

With the seams between the insulator 6 and the electrode elements 2, repeated expansion and contraction may create gaps at the seams and cause the wire 4 to be exposed to air. This may cause oxidation of the wire 4 disposed inside and may lead to degraded conductivity. In the surface electrode 10g according to Embodiment 9, on the other hand, the wire 4 is seamlessly covered with the insulator 6 therearound. With less seams in the covering, the wire 4 is less exposed to air and this can make the material resistant to oxidation.

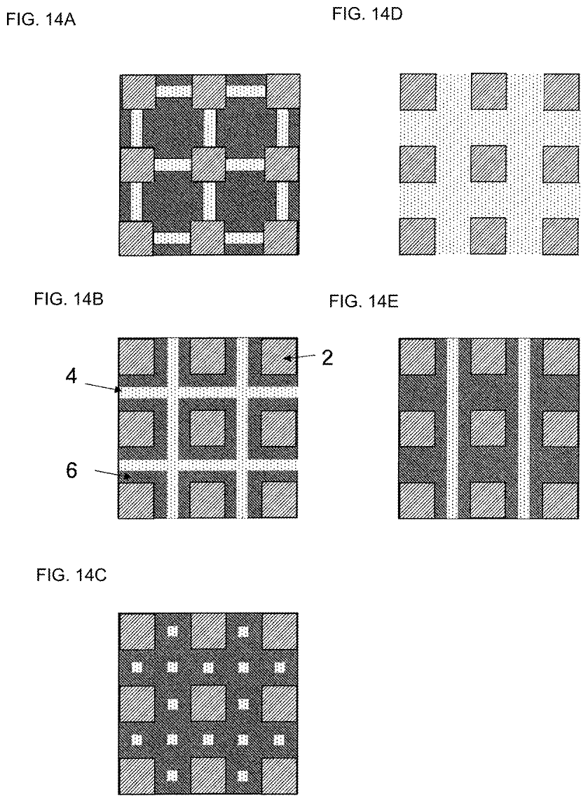

FIG. 14A to FIG. 14E are schematic bottom views illustrating various patterns of a planar arrangement of the wire 4.

### Embodiment 10

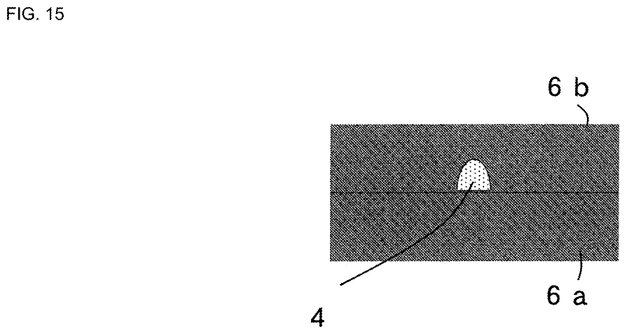

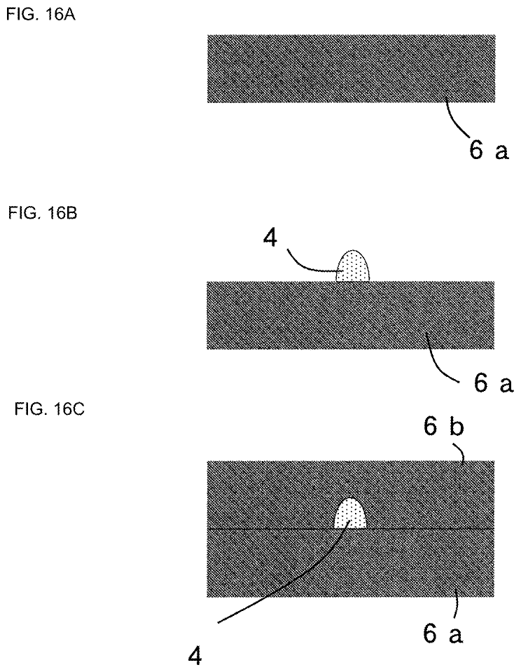

FIG. 15 is a schematic cross-sectional view illustrating a cross-sectional structure of insulators 6a and 6b and the wire 4 in a surface electrode according to Embodiment 10. FIG. 16 is a schematic cross-sectional view illustrating a process of manufacturing the cross-sectional structure of the insulators 6a and 6b and the wire 4 illustrated in FIG. 15.

The surface electrode according to Embodiment 10 includes the first and second insulators 6a and 6b having a two-layer structure that holds the wire 4 sandwiched between layers.

The cross-sectional structure of the first and second insulators 6a and 6b and the wire 4 is made as follows:
(1) The first insulator 6a is prepared (FIG. 16A);
(2) The wire 4 is placed on the surface of the first insulator 6a (FIG. 16B). For example, the wire 4 may be in liquid form. The wire 4 placed is fixed. For example, when the wire 4 is a paramagnetic, soft magnetic, or ferromagnetic wire, the wire 4 can be fixed in any planar pattern with strong magnetic force from the back side of the first insulator 6a; and
(3) The second insulator 6b is placed over the wire 4 fixed as described above (FIG. 16C).

The cross-sectional structure of the first and second insulators 6a and 6b and the wire 4 is thus obtained.

In the cross-sectional structure of the first and second insulators 6a and 6b and the wire 4, the second insulator 6b is placed to conform to the surface shape of the wire 4. This can reduce gaps between the wire 4 and the first and second insulators 6a and 6b, and can prevent entry of sweat and water into the wire 4 from outside.

### Embodiment 11

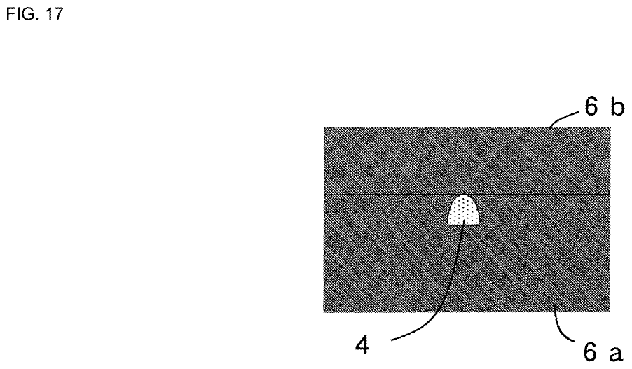

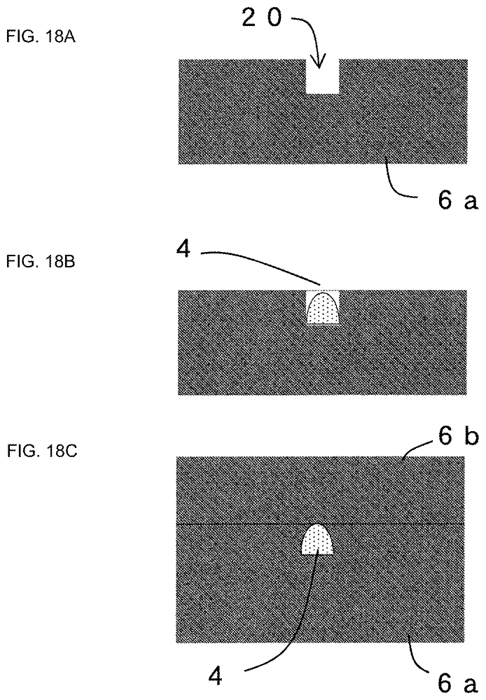

FIG. 17 is a schematic cross-sectional view illustrating a cross-sectional structure of the first and second insulators 6a and 6b and the wire 4 in a surface electrode according to Embodiment 11. FIG. 18 is a schematic cross-sectional view illustrating a process of manufacturing the cross-sectional structure of the first and second insulators 6a and 6b and the wire 4 illustrated in FIG. 17.

The surface electrode according to Embodiment 11 includes the first and second insulators 6a and 6b having a two-layer structure that holds the wire 4 sandwiched between layers.

The cross-sectional structure of the first and second insulators 6a and 6b and the wire 4 is made as follows:
(1) The first insulator 6a having a groove 20 in a surface thereof is prepared (FIG. 18A);
(2) The wire 4 is placed along the groove 20 in the surface of the first insulator 6a (FIG. 18B). For example, the wire 4 may be in liquid form. The wire 4 placed is fixed. In this case, the wire 4, which extends along the groove 20, does not move easily. When a liquid material, such as a liquid metal, is used to form the wire 4, the material is applied to fit within the height of the groove 20. The wire 4 can thus be sealed, with its shape maintained; and
(3) The second insulator 6b is placed over the wire 4 fixed as described above (FIG. 18C).

The cross-sectional structure of the first and second insulators 6a and 6b and the wire 4 is thus obtained.

In the cross-sectional structure of the first and second insulators 6a and 6b and the wire 4, the insulators are joined together, with one being filled in a recess in the other. This can increase the contact area, and can prevent entry of sweat and water into the wire from outside.

### Embodiment 12

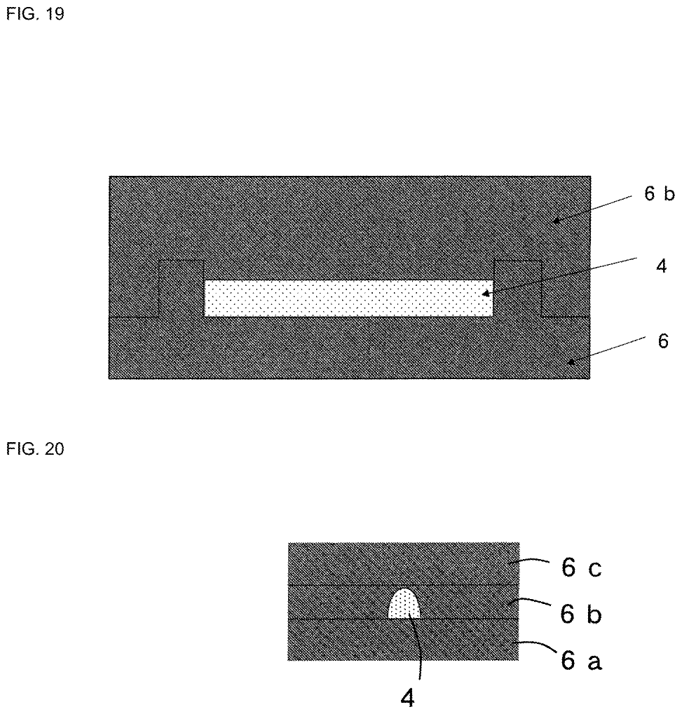

FIG. 19 is a schematic cross-sectional view illustrating a cross-sectional structure of the first and second insulators 6a and 6b and the wire 4 in a surface electrode according to Embodiment 12, as viewed in a direction perpendicular to the longitudinal direction of the wire 4.

The cross-sectional structure may be formed in the same way as above. That is, after the wire 4 is placed in a recess in the first insulator 6a, the second insulator 6b is placed over the wire 4.

This can increase the contact area between the insulators, and can more effectively prevent entry of sweat and water into the wire 4 from outside.

### Embodiment 13

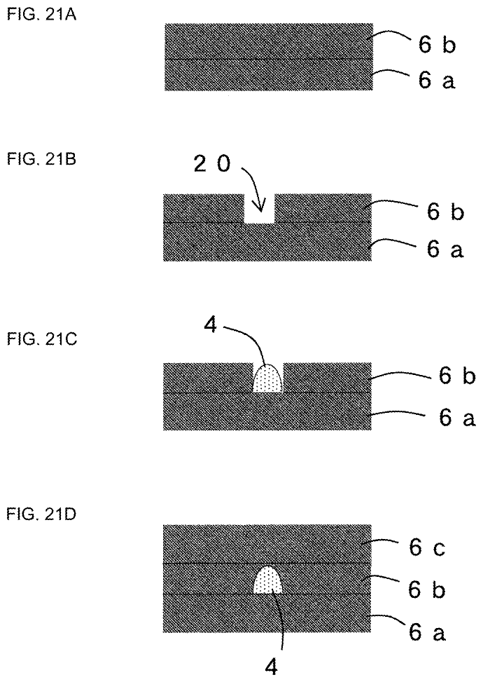

FIG. 20 is a schematic cross-sectional view illustrating a cross-sectional structure of the first to third insulators 6a, 6b, and 6c and the wire 4 in a surface electrode according to Embodiment 13. FIGS. 21A to 21D are schematic cross-sectional views illustrating a process of manufacturing the cross-sectional structure of the first to third insulators 6a, 6b, and 6c and the wire 4 illustrated in FIG. 20.

The surface electrode according to Embodiment 13 includes the first to third insulators 6a, 6b, and 6c having a three-layer structure that holds the wire 4, with upper and lower surfaces a layer including therein the wire 4 sandwiched between the other layers.

The cross-sectional structure of the first to third insulators 6a, 6b, and 6c and the wire 4 is made as follows:
(1) The second insulator 6b is placed on the first insulator 6a (FIG. 21A);
(2) The groove 20 is formed in the second insulator 6b (FIG. 21B);
(3) The wire 4 is placed along the groove 20 (FIG. 21C). For example, the wire 4 may be in liquid form. The wire 4 placed is fixed. In this case, the wire 4, which extends along the groove 20, does not move easily. When a liquid material, such as a liquid metal, is used to form the wire 4, the material is applied to fit within the height of the groove 20. The wire 4 can thus be sealed, with its shape maintained; and
(4) The third insulator 6c is placed over the wire 4 fixed as described above (FIG. 21D).

The cross-sectional structure of the first to third insulators 6a, 6b, and 6c and the wire 4 is thus obtained.

The first to third insulators 6a, 6b, and 6c and the wire 4 have a cross-sectional structure in which the insulators 6a, 6b, and 6c are formed in three layers to increase the contact area between the insulators. This can enhance strength against changes in the measured surface.

### Embodiment 14

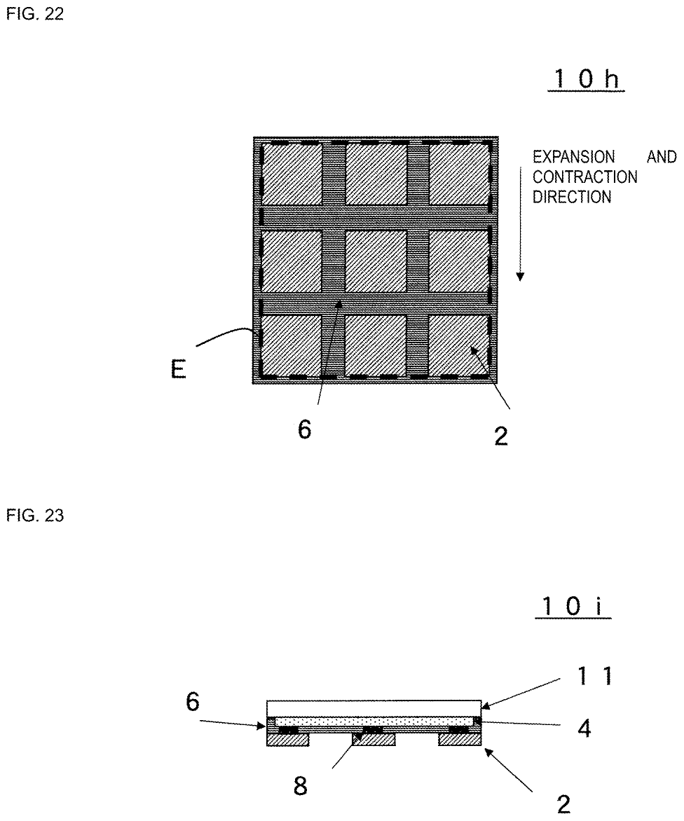

FIG. 22 is a schematic bottom view illustrating an arrangement of the electrode elements 2 and the insulator 6 in a surface electrode 10h according to Embodiment 14.

The surface electrode 10h according to Embodiment 14 is configured in such a way that, in a range E defined by the outer edge of the electrode elements 2 in plan view of the measured surface of the object to be measured, an area occupied by the electrode elements 2 is greater than an area outside the electrode elements 2. The distance between adjacent ones of the electrode elements 2 may be greater than a thickness of any of the electrode elements 2.

When the shape of the measured surface changes, the configuration, described above, allows efficient acquisition of signals obtainable from the area where the electrode elements 2 are present. When a sufficiently stretchable material is used, stress applied to the joint of the electrode elements 2 and the insulator 6 by changes in the shape of the measured surface can be reduced, and the performance of following the shape changes can be further improved. When the surface of a living body is measured, noise caused by shape changes is reduced. Also, when electric stimulation is applied to a living body, it is possible to reduce changes in electric pulse actually applied in the living body.

Also, for example, with the wire 4 being softer than the electrode elements 2, stress generated by changes in the shape of the electrode elements 2 can be reduced. This can reduce noise caused by the stress. Examples of the noise include deformation of the electrode elements 2, and positional displacement between the electrode elements 2 and the measured surface.

The wire 4 may be more stretchable than the electrode elements 2.

The meaning of “A is more stretchable than B” and how stretchability is measured will now be described. First, “being stretchable” means being elastically deformable. Of various types of deformation caused by applying force to an object, elastic deformation refers to a deformation which allows the object to return to its original shape once the applied force is removed. Note that when force exceeding the range of elastic deformation is applied to an object and the object does not return to its original shape after removal of the applied force, the deformation is referred to as plastic deformation, not as elastic deformation. “A is more stretchable than B” means that the elastically-deformable length of A is greater than that of B (A>B) or the tensile modulus of elasticity of B is greater than that of A (B>A).

The tensile modulus of elasticity being “B>A” means that when the same tension is applied to B and A, the length by which A is stretched is greater than the length by which B is stretched.

This is measured by applying the same tension to A and B of equal length, in the length direction, to determine which of A and B is longer. The longer of A and B is more stretchable than the other.

When the wire 4 is more stretchable than the electrode elements 2, stress generated by changes in the shape of the electrode elements 2, as the shape of the measured surface changes, can be reduced by the wire 4 being softer than the electrode elements 2. This can reduce noise caused by the stress. Examples of the noise include deformation of the electrode elements 2, and positional displacement between the electrode elements 2 and the measured surface. When the surface of a living body is measured, noise caused by shape changes is reduced. Also, when electric stimulation is applied to a living body, it is possible to reduce changes in electric pulse actually applied in the living body.

### Embodiment 15

FIG. 23 is a schematic cross-sectional view illustrating a cross-sectional structure of a surface electrode 10i according to Embodiment 15.

The surface electrode 10i according to Embodiment 15 includes the substrate 11 on a surface opposite the surface to be in contact with the measured surface. The substrate 11 is softer than the electrode elements 2.

When the shape of the measured surface changes, the substrate 11 allows expansion and contraction in the planar direction while distributing stress on other layers. When the surface of a living body is measured as an object to be measured, noise caused by shape changes is reduced. Also, when electric stimulation is applied to a living body, it is possible to reduce changes in electric pulse actually applied in the living body.

The substrate 11 may have harder characteristics than the wire 4. In this case, when the shape of the measured surface changes, the substrate 11 being harder than the wire 4 can limit the degree of bending. This can prevent breakage of the wire 4.

The meaning of “A is harder than B” is as follows.

Assume that the same pressure is applied to the midpoints of A and B each fixed at both ends. “A is harder than B” is true when the radius of curvature of A is greater than that of B (A>B). The radius of curvature is the radius of a circle that approximates a curve of an object bent by bending force. Here, the radius of curvature is a radius obtained when a curve formed as a result of deformation caused by pressure applied to the midpoint of the object is regarded as a circumference.

The radius of curvature can be measured, for example, by photographing the deformation, drawing a circle from the circumference on the basis of the photograph, determining the radius at the edge of the circle as the radius of curvature, and comparing two radii of curvature to determine the harder one.

The substrate 11 may have softer characteristics than the wire 4. In this case, when the shape of the measured surface changes, the wire 4 can closely follow excessive shape changes, because of the substrate 11 being softer than the wire 4.

The via 8 for electrically connecting the wire 4 to the electrode element 2 is provided between the wire 4 and the electrode element 2. For example, the via 8 is made of a mixture of a conductive material and a resin material.

Since the wire 4 is connected to the electrode element 2, with the via 8 therebetween, the wire 4 can expand and contract to follow the measured surface in the case of occurrence of excessive changes in the measured surface. This can prevent breakage between the electrode element 2 and the wire 4.

In the mixture of the conductive material and the resin material forming the via 8, the conductive material may be a carbon-based conductive material.

Adjacent ones of the electrode elements 2 may be connected by a dense planar insulator. That is, gaps between adjacent ones of the electrode elements 2 are filled with the dense planar insulator. When the surface electrode 10i is used on the surface of a living body, the entry of substances, such as sweat from the skin, into the wire 4 can be prevented. Thus, since there are no gaps between adjacent ones of the electrode elements 2, it is possible to reduce changes in electrical resistance that would occur as a result of entry of external substances in the presence of such gaps.

The wire 4 may be disposed on the side of the electrode elements 2 opposite the measured surface. That is, the wire 4 may be disposed on the back side of the electrode elements, or on the upper side in the Z axis direction.

### Embodiment 16

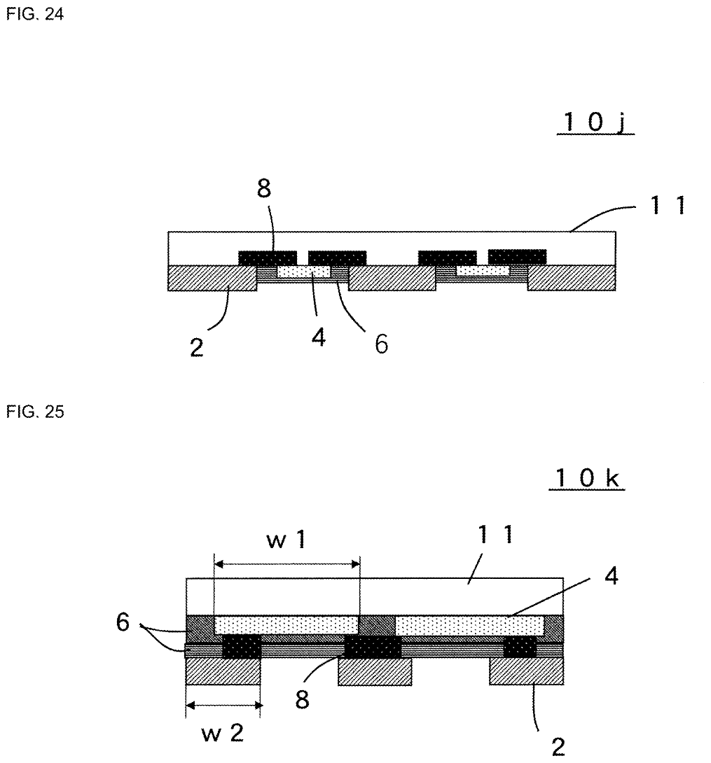

FIG. 24 is a schematic cross-sectional view illustrating a cross-sectional structure of a surface electrode 10j according to Embodiment 16.

In the surface electrode 10j according to Embodiment 16, the wire 4 is disposed between adjacent ones of the electrode elements 2, that is, disposed in the in-plane direction.

In this case, the wire 4 between the electrode elements 2 expands and contracts as the measured surface expands and contract. The electrode elements 2 are connected to the wire 4, with the via 8 interposed therebetween in the in-plane direction.

### Embodiment 17

FIG. 25 is a schematic cross-sectional view illustrating a cross-sectional structure of a surface electrode 10k according to Embodiment 17. FIG. 26 is a schematic cross-sectional view illustrating a cross-sectional structure of a surface electrode 101 according to another example of Embodiment 17.

In the surface electrode 10k according to Embodiment 17, as illustrated in FIG. 25, a width w1 of the wire 4 is greater than a width w2 of the electrode elements 2 in the planar direction of the surface. In the example illustrated in FIG. 26, the wire 4 is disposed over substantially the entire surface, and the width w1 of the wire 4 is obviously greater than the width w2 of the electrode elements 2.

When the shape of the measured surface changes, the wire 4, which has the width w1 greater the width w2 of the electrode elements 2, can be prevented from breaking.

The wire 4 may extend outward beyond the outside diameter of the via 8 in the planar direction. Thus, although the wire 4 expands and contracts as the measured surface expands and contracts, the resulting changes in contact area between the wire 4 and the via 8 can be reduced.

### Embodiment 18

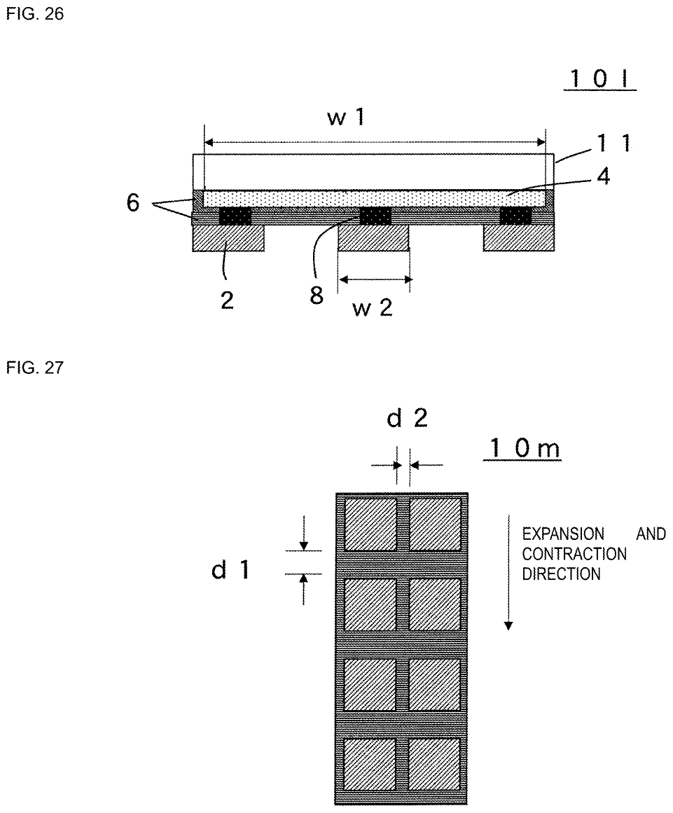

FIG. 27 is a schematic bottom view illustrating an arrangement of the electrode elements 2 and the insulator 6 in a surface electrode 10m according to Embodiment 18.

In the surface electrode 10m according to Embodiment 18, the distances between adjacent ones of the plurality of electrode elements 2 spaced from each other are defined as a first distance d1 and a second distance d2. The first distance d1 is the distance between adjacent ones of the electrode elements 2 in the expansion and contraction direction in the plane of the measured surface, and the second distance d2 is the distance between adjacent ones of the electrode elements 2 in the direction perpendicular to the expansion and contraction direction. In this case, at least the first distance d1 is longer than the second distance d2.

When the first distance d1 is longer than the second distance d2, the performance of following the expansion and contraction of the measured surface can be improved.

### Embodiment 19

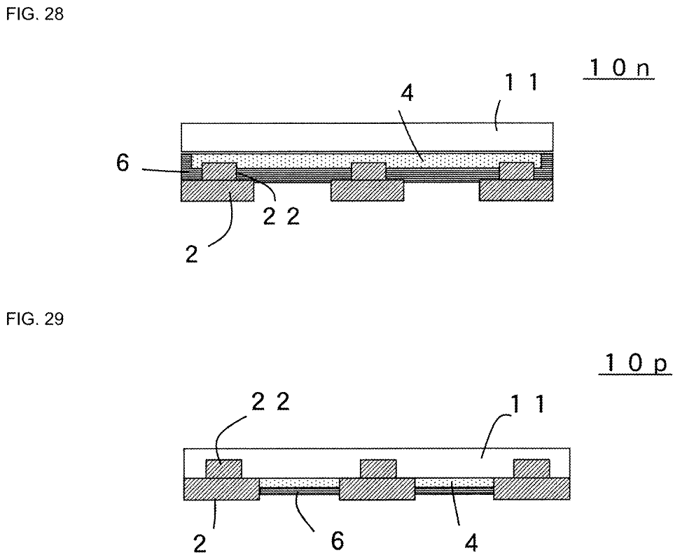

FIG. 28 is a schematic cross-sectional view illustrating a cross-sectional structure of a surface electrode 10n according to Embodiment 19. FIG. 29 is a schematic cross-sectional view illustrating a cross-sectional structure of a surface electrode 10p according to another example of Embodiment 19.

In the surface electrode 10n according to Embodiment 19, the electrode elements 2 each have a protrusion 22 protruding in a direction orthogonal to the first surface. In the example illustrated in FIG. 28, the protrusion 22 is embedded in the insulator 6 or in the wire 4. In the example illustrated in FIG. 29, the protrusion 22 is embedded in the substrate 11.

In the example illustrated in FIG. 28, the protrusion 22 of the electrode element 2 makes an electrical connection to the wire 4. In the example illustrated in FIG. 29, adjacent ones of the electrode elements 2 are electrically connected by the wire 4 therebetween.

When the measured surface expands and contracts, the configuration described above allows the protrusions 22 on the electrode elements 2 to serve as an anchor and prevent the electrode elements 2 from coming off.

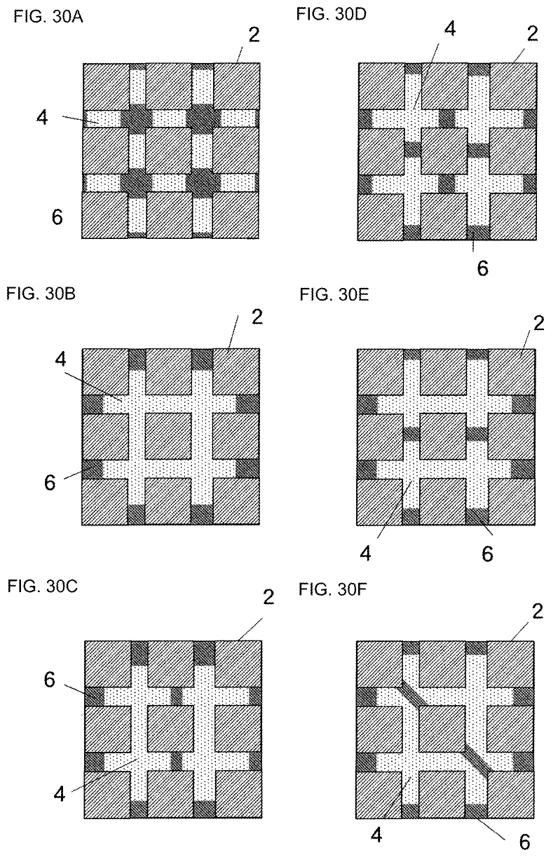

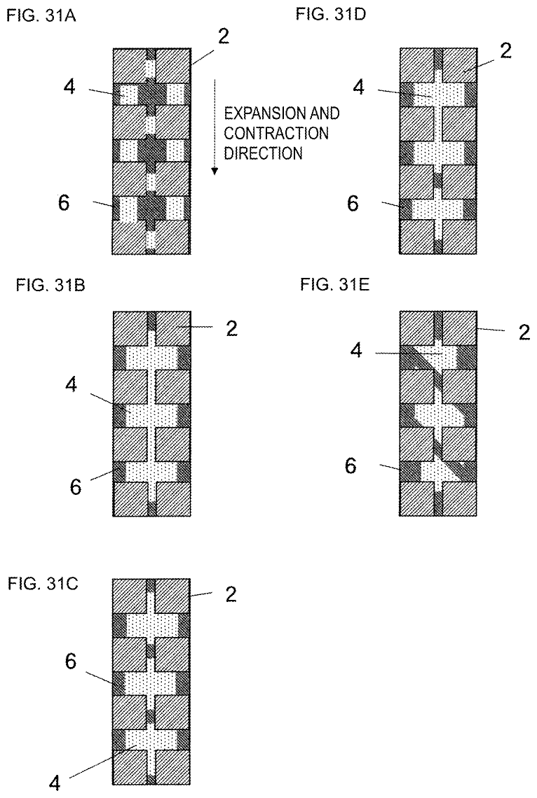

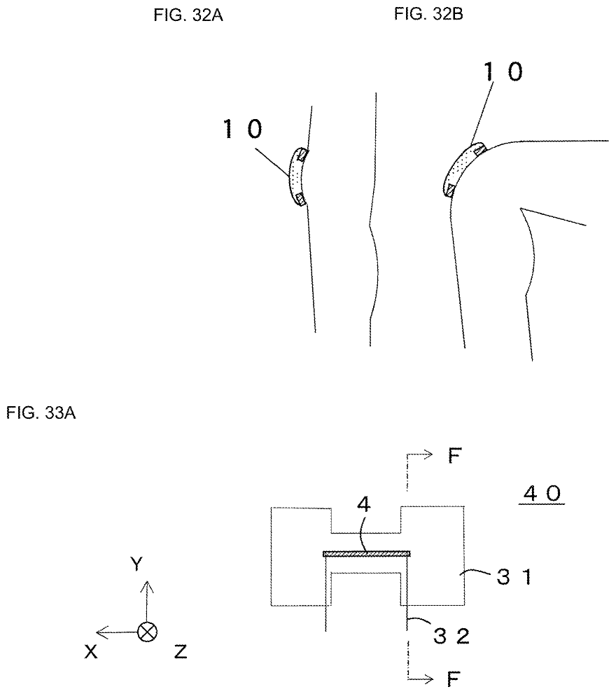

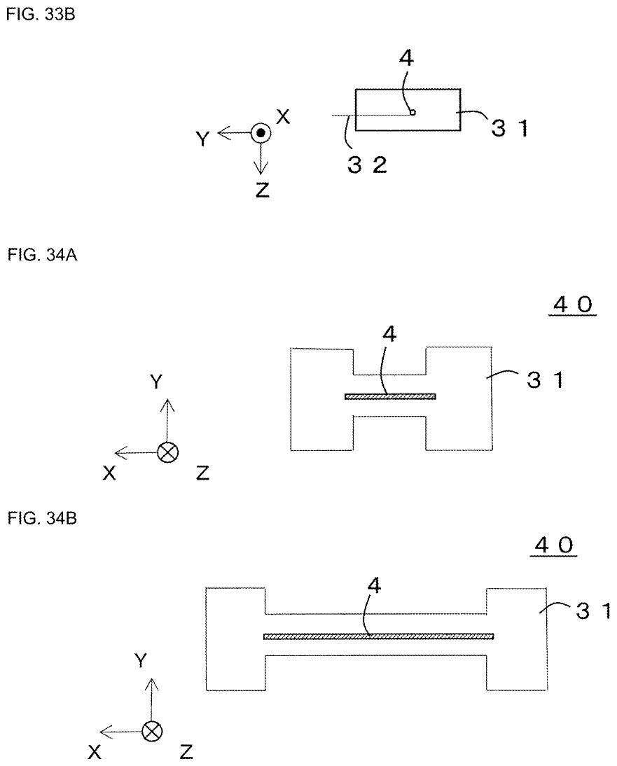

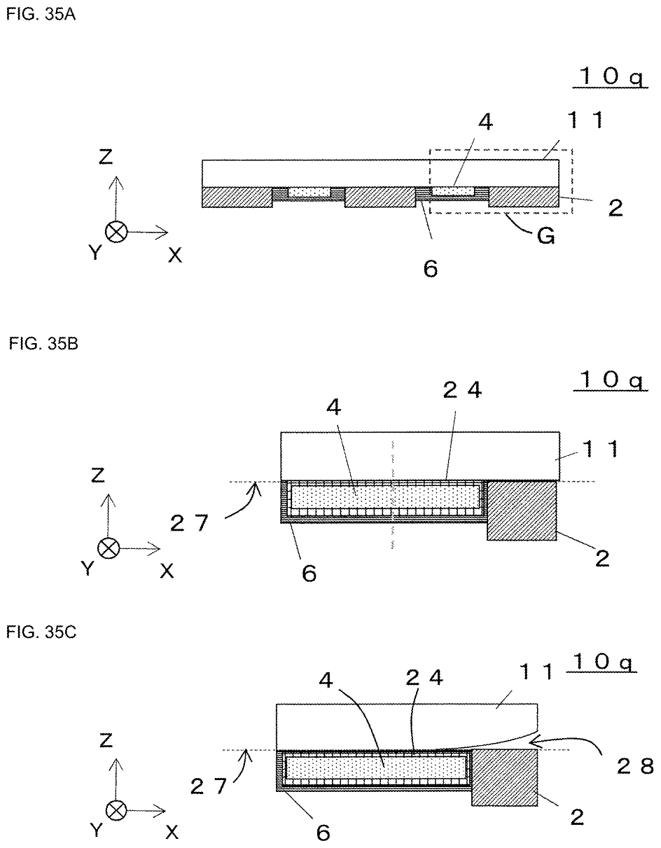

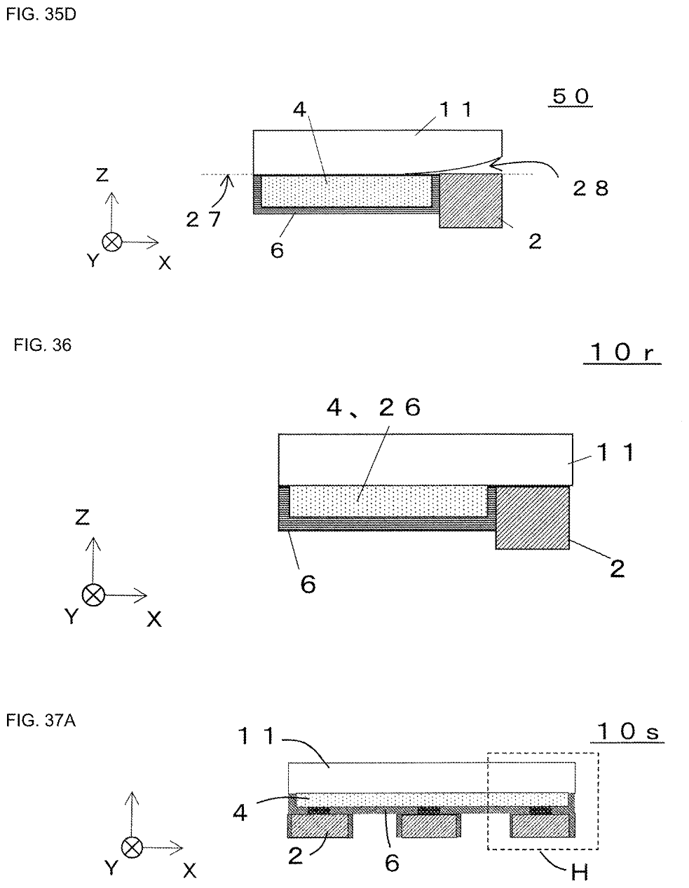

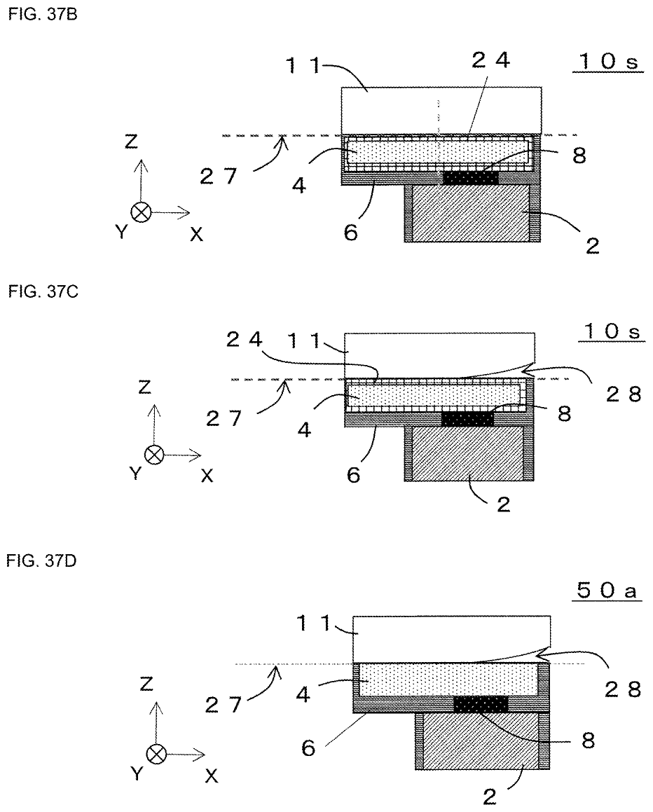

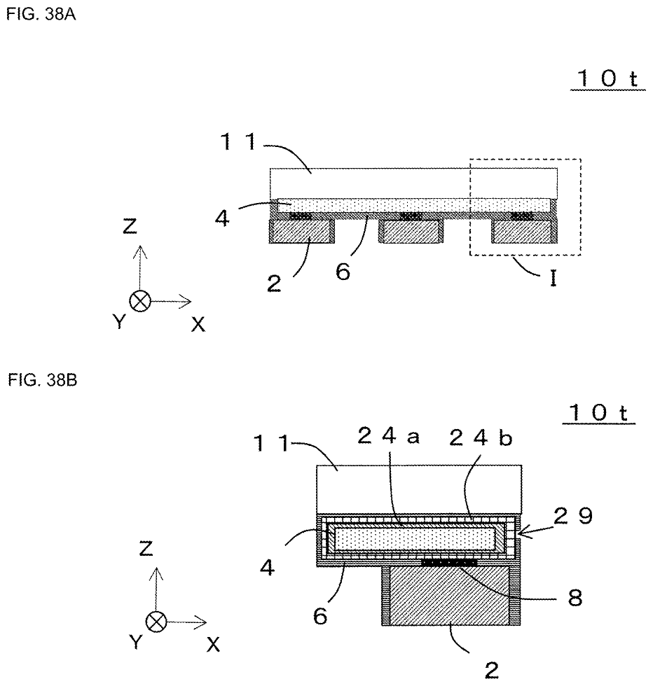

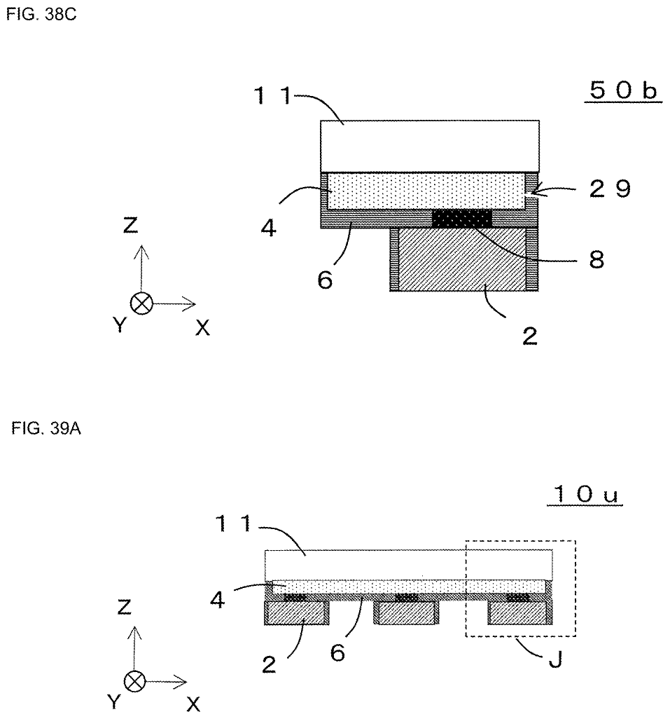

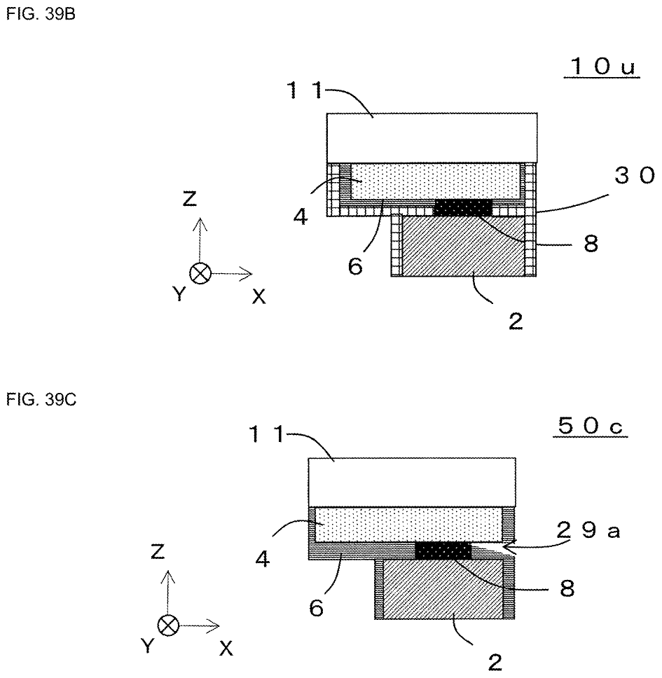

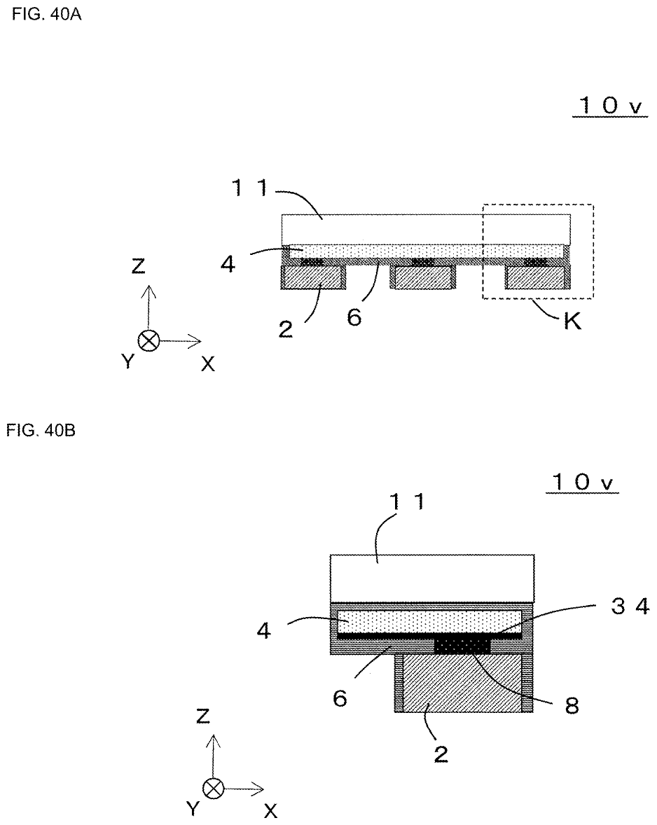

FIG. 30A to FIG. 30F are schematic perspective views illustrating various patterns of a planar arrangement of the wire 4.

FIG. 3IA to FIG. 3IE are schematic bottom views illustrating various patterns of a planar arrangement of the wire 4 where a distance between adjacent ones of the electrode elements in an expansion and contraction direction differs from that in a direction perpendicular to the expansion and contraction direction.

FIG. 32A is a schematic diagram illustrating an example in which the surface electrode 10 according to Embodiment 1 is attached to a knee of a leg, and FIG. 32B is a schematic diagram illustrating a bent position of the knee illustrated in FIG. 32A.

For example, the surface electrode may be attached with a fastening belt or a gel to a human (or person's) knee, which is an object to be measured.

Noise Generated in Wire

FIG. 33A is a plan view of a surface electrode 40 including the wire 4 for a test, and FIG. 33B is a schematic cross-sectional view illustrating a cross-sectional structure of the surface electrode 40, as viewed in the direction F-F of FIG. 33A. FIG. 34A is a plan view of the same surface electrode 40 as that in FIG. 33A, and FIG. 34B is a plan view illustrating the surface electrode of FIG. 34A deformed by tensile force applied thereto in the X direction.

With reference to FIG. 33A to FIG. 34B, noise generated in the sealed wire 4 under tensile force will be described. As illustrated in FIG. 33A, the surface electrode 40 having an H-shape is prepared. The surface electrode 40 includes, for example, the wire 4 sealed with a silicon resin 31 and a pair of conductors 32 connected to the wire 4. The impedance of the wire 4 can be measured by taking out the pair of conductors connected to the wire 4. The conductors 32 are disposed at both end portions of the wire 4 in a first direction (X direction) in which the wire 4 extends. After an impedance R₀ in the initial state is measured, a tensile force is applied to the surface electrode 40 in the first direction (X direction) in such a way that an expansion ratio of 100% is reached. Then, an impedance R is measured at an expansion ratio of 100%. An expansion ratio of 100% means that the wire 4 is 2X in length, where X is the length from one end to the other end of the wire 4 in the first direction (X direction) under no tensile force. On the other hand, an expansion ratio of 0% means that the wire 4 is X in length from one end to the other end thereof in the first direction (X direction).

Table 1 compares resistance change ratios at an expansion ratio of 100% between surface electrodes, one including a wire made of a conductive paste (e.g., Ag paste) and the other including a wire made of a liquid metal. Assume that the impedance R₀ at an expansion ratio of 0% (length X) is 1Ω in both the surface electrodes. When subjected to a tensile force until an expansion ratio of 100% (length 2X) was achieved, the surface electrode including the wire made of a conductive paste had the impedance R (resistance: 130Ω) 130 times the impedance R₀, whereas the surface electrode including the wire made of a liquid metal had the impedance R (resistance: 3Ω) about 3 times the impedance R₀. This indicates that the wire made of a liquid metal generates less noise than the wire made of a conductive paste.

The wire 4 may be made of an electrolyte solution, and does not necessarily need to be made of a liquid metal. The wire 4 may be made of an aqueous solution containing metal powder, or may be made of an aqueous solution containing metal coated with conductive resin.

TABLE 1

| | Resistance change ratio at |
|---|---|
| | expansion ratio of 100%: |
| Wire | (R-R₀)/R₀ × 100 [ %] |
| Conductive paste | 130 |
| Liquid metal | 3 |

In Table 1, R₀ is the impedance (resistance) of the wire before being stretched (expansion ratio of 0%), and R is the impedance (resistance) of the wire being stretched (expansion ratio of 100%).

The definition of the expansion ratio is not limited to this. For example, when there are two adjacent electrode elements, with a wire therebetween, an expansion ratio of 100% may mean that the distance between the two electrode elements is 2X, where X is the distance from one end to the other end of each electrode element under no external pressure.

### Embodiment 20

FIG. 35A is a schematic cross-sectional view illustrating a cross-sectional structure of a surface electrode 10q according to Embodiment 20. FIG. 35B is an enlarged cross-sectional view of a region G indicated in FIG. 35A by a dotted line, which encloses one electrode element 2 at an end portion of the surface electrode 10q. FIG. 35C is an enlarged cross-sectional view illustrating a separation 28 at the end portion of the surface electrode 10q illustrated in FIG. 35B. FIG. 35D is an enlarged cross-sectional view illustrating the separation 28 in a surface electrode 50 of a reference example which does not include a sealing portion for sealing the wire.

As illustrated in FIG. 35B, the surface electrode 10q according to Embodiment 20 differs from the surface electrode according to Embodiment 1 in that the surface electrode 10q includes the sealing portion 24 for sealing the wire 4. The sealing portion 24 may have conductivity to allow conduction between the wire 4 and the electrode element 2. In the surface electrode 10q, as illustrated in FIG. 35C, even when the separation 28 occurs at a seam 27 in resin between the substrate 11 and the electrode element 2 and the wire 4, leakage from the wire 4 does not occur, because the wire 4 is sealed with the sealing portion 24. In the surface electrode 50 of the reference example which does not include a sealing portion, as illustrated in FIG. 35D, the separation 28 between the substrate 11 and the electrode element 2 and the wire 4 may cause leakage from the wire 4.

Sealing Portion

The sealing portion 24 is required to simply seal the perimeter of the wire 4. Although the sealing portion 24 illustrated in FIG. 35B seals the entire perimeter of the wire 4, the configuration is not limited to this. The sealing portion 24 may seal the wire 4 on a predetermined unit basis. For example, the entire wire 4 on the surface may be covered with a single integral sealing portion. A plurality of sealing portions may seal the wire on a row-by-row or column-by-column basis. A plurality of sealing portions may seal the wire on a unit area basis. When a plurality of sealing portions seal the wire on a region-by-region basis, it is simply required that conduction be achieved between adjacent ones of the sealing portions.

The sealing portion 24 may be made of a stretchable resin, such as elastomer, PDMS, or PVP, or may be made of hydrogel. The sealing portion 24 may be made of a fibrous material, such as polyurethane, or may be made of tungsten oxide, copper, or gallium oxide (Ga₂O₃). The sealing portion 24 is not limited to one that is formed by a single component. For example, the sealing portion 24 may be made of a composite of materials, such as resin and copper. The sealing portion 24 may be an insulating portion, or may have conductivity to allow conduction with the electrode element. As described below, the sealing portion may include a first sealing portion on the inner side and a second sealing portion on the outer side. In this case, the first sealing portion may be a conductive sealing portion, and the second sealing portion may be an insulating sealing portion. The first sealing portion on the inner side may be a solid wire. The second sealing portion on the outer side may be an insulator. The sealing portion may also be referred to as a supporter or a protective layer, depending on the function.

The sealing portion 24 may contain a porous material. For example, the porous material may be a sponge containing resin. With the sealing portion 24 containing a porous material, the porous material retains liquid forming the wire 4. The porous material, which is solid, makes the wire 4 resistant to deformation and this can reduce noise. The porous material can effectively reduce deformation of the wire 4 particularly when the surface electrode 10q is deformed. Beside resin, the porous material may contain cloth or metal. The porous material may be, for example, a nonwoven fabric.

### Embodiment 21

FIG. 36 is a schematic cross-sectional view illustrating a cross-sectional structure of a surface electrode 10r according to Embodiment 21.

As illustrated in FIG. 36, the surface electrode 10r according to Embodiment 21 differs from the surface electrode according to Embodiment 1 in that the wire 4 contains a porous material 26. The porous material 26 is, for example, a sponge containing resin. With the wire 4 containing a porous material, the porous material retains liquid forming the wire 4. The porous material, which is solid, makes the wire 4 resistant to deformation and this can reduce noise. The porous material can effectively reduce deformation of the wire 4 particularly when the surface electrode 10r is deformed. Beside resin, the porous material 26 may contain cloth or metal. The porous material 26 may be, for example, a nonwoven fabric.

### Embodiment 22

FIG. 37A is a schematic cross-sectional view illustrating a cross-sectional structure of a surface electrode 10s according to Embodiment 11. FIG. 37B is an enlarged cross-sectional view of a region H indicated in FIG. 37A by a dotted line, which encloses one electrode element 2 at an end portion of the surface electrode 10s. FIG. 37C is an enlarged cross-sectional view illustrating the separation 28 at the end portion of the surface electrode 10s illustrated in FIG. 37B. FIG. 37D is an enlarged cross-sectional view illustrating the separation 28 in a surface electrode 50a of a reference example which does not include a sealing portion for sealing the wire 4.

As illustrated in FIG. 37B, the surface electrode 10s according to Embodiment 22 differs from the surface electrode according to Embodiment 1 in that the surface electrode 10s includes the sealing portion 24 for sealing the wire 4. In the surface electrode 10s, as illustrated in FIG. 37C, even when the separation 28 occurs at the seam 27 in resin between the substrate 11 and the electrode element 2 and the wire 4, leakage from the wire 4 does not occur, because the wire 4 is sealed with the sealing portion 24. In the surface electrode 50a of the reference example which does not include a sealing portion, as illustrated in FIG. 37D, the separation 28 between the substrate 11 and the electrode element 2 and the wire 4 may cause leakage from the wire 4.

### Embodiment 23

FIG. 38A is a schematic cross-sectional view illustrating a cross-sectional structure of a surface electrode 10t according to Embodiment 23. FIG. 38B is an enlarged cross-sectional view of a region I indicated in FIG. 38A by a dotted line, which encloses one electrode element 2 at an end portion of the surface electrode 10t. FIG. 38B illustrates a crack 29 formed at the end portion of the surface electrode 10t. FIG. 38C is an enlarged cross-sectional view illustrating a crack in a surface electrode of a reference example which does not include a sealing portion for sealing the wire.

As illustrated in FIG. 38B, the surface electrode 10t according to Embodiment 23 differs from the surface electrode according to Embodiment 1 in that the surface electrode 10t includes a first sealing portion 24a for sealing the wire 4 and a second sealing portion 24b disposed outside the first sealing portion 24a. The first sealing portion 24a and the second sealing portion 24b function as a solid wire. In the surface electrode 10t, even if the insulator 6 is broken by sudden expansion and contraction or external force, the first sealing portion 24a and the second sealing portion 24b can reduce leakage of liquid from the wiring 4.

The first sealing portion 24a is disposed on the inner side of the surface electrode 10t, and the second sealing portion 24b is disposed outside the first sealing portion 24a. The first sealing portion 24a and the second sealing portion 24b may have different moduli of elasticity. For example, if the relation “modulus of elasticity of the first sealing portion 24a>modulus of elasticity of the second sealing portion 24b” holds true, then even if the surface electrode 10t is subjected to pressure, the resulting noise can be reduced. This is because the second sealing portion 24a deforms to absorb the pressure, whereas the first sealing portion 24a is more resistant to deformation than the second sealing portion 24b.

On the other hand, if the relation “modulus of elasticity of the first sealing portion 24a<modulus of elasticity of the second sealing portion 24b” holds true, then even if the second sealing portion 24b is damaged by pressure applied to the surface electrode 10t, the damage to the second sealing portion 24b does not significantly affect the first sealing portion 24a, which is more deformable. With the first sealing portion 24a resistant to damage, the leakage of liquid forming the wire to the outside is reduced. This can prevent the occurrence of noise caused by leakage to the outside.

The first sealing portion 24a and the second sealing portion 24b may be separate and movable with respect to each other. In this case, even if the second sealing portion 24b is damaged by pressure applied to the surface electrode 10t, the damage to the second sealing portion 24a does not significantly affect the first sealing portion 24a, because the first sealing portion 24a and the second sealing portion 24b are movable with respect to each other.

The first sealing portion 24a and the second sealing portion 24b may have different colors. The different colors allow the user to identify any damage to the second sealing portion 24b. This can prevent the liquid forming the wire from leaking to the outside.

### Embodiment 24

FIG. 39A is a schematic cross-sectional view illustrating a cross-sectional structure of a surface electrode 10u according to Embodiment 13. FIG. 39B is an enlarged cross-sectional view of a region J indicated in FIG. 39A by a dotted line, which encloses one electrode element 2 at an end portion of the surface electrode 10u. FIG. 39C is an enlarged cross-sectional view illustrating a crack 29a in a surface electrode 50c of a reference example which does not include a sealing portion serving as a protective layer covering an outer side portion of the insulator.

As illustrated in FIG. 39B, the surface electrode 10u according to Embodiment 24 differs from the surface electrode according to Embodiment 1 in that the surface electrode 10u includes a sealing portion 30 serving as a protective layer covering the outer side portion of the insulator 6. In the surface electrode 10u, even if the insulator 6 is broken by sudden expansion and contraction or external force, the sealing portion 30 serving as a protective layer covering the outer side portion of the insulator 6 can reduce leakage of liquid from the wire.

### Embodiment 25

FIG. 40A is a schematic cross-sectional view illustrating a cross-sectional structure of a surface electrode 10v according to Embodiment 14. FIG. 40B is an enlarged cross-sectional view of a region K indicated in FIG. 40A by a dotted line, which encloses one electrode element 2 at an end portion of the surface electrode 10v.

As illustrated in FIG. 40B, the surface electrode 10v according to Embodiment 25 differs from the surface electrode according to Embodiment 1 in that the surface electrode 10v includes a magnet 34 near the electrode element 2. In this case, the wire may contain a ferromagnetic material, such as Fe, Ni, or Co. The magnet 34 allows the wire 4 containing a ferromagnetic material to be held near wiring of the surface electrode 10v. This can reduce the occurrence of noise even when the shape of the surface electrode 10v changes. In plan view of the magnet 34 viewed in a direction normal to the upper surface of the substrate 11, the magnet 34 preferably overlaps the electrode element 2.

Higher wettability between the wire and the electrode elements can provide better conductivity. The wettability of the electrode elements can be improved, for example, by making the surface roughness of the electrode elements as small as 1 μm or less. With a conductive liquid layer (referred to as “slip layer”), such as an electrolyte layer, between the wire and the electrode elements, the wettability between the wire and the electrode elements can be improved. For improved wettability, for example, the degree of humidity between the wire and the electrode elements is preferably a relative humidity of greater than or equal to 50%, and more preferably greater than or equal to 75%.

According to Aspect 25, in the surface electrode of Aspect 1, the surface electrode may further include a magnet, the wire may contain a liquid and a ferromagnetic material, and in plan view of the magnet viewed in a direction normal to the first surface, the magnet may overlap at least one of the electrode elements.

According to Aspect 26, in the surface electrode of Aspect 1, the surface electrode may further include a sealing portion configured to seal the wire, and the sealing portion may include a first sealing portion and a second sealing portion disposed outside the first sealing portion.

According to Aspect 27, in the surface electrode of Aspect 26, a modulus of elasticity of the first sealing portion may be greater than a modulus of elasticity of the second sealing portion.

According to Aspect 28, in the surface electrode of Aspect 26, a modulus of elasticity of the second sealing portion may be greater than a modulus of elasticity of the first sealing portion.

According to Aspect 29, in the surface electrode of Aspect 26, the wire may contain a liquid, and the first sealing portion may contain a porous material.

According to Aspect 30, in the surface electrode of Aspect 1, the wire may contain a liquid, and the wire may contain a porous material.

The present disclosure includes appropriate combinations of any of the various embodiments and/or examples described above, and achieves advantageous effects of the corresponding embodiments and/or examples.

The surface electrode according to the present invention can be used as a biomedical electrode that follows changes of a body surface and causes less noise.

What is claimed is:

1. A surface electrode having a first surface, the surface electrode comprising:
a plurality of electrode elements on the first surface and spaced from each other in a manner so as to be configured to contact a measured surface of an object to be measured;
a stretchable wire electrically connecting the plurality of electrode elements; and
a stretchable insulator covering a side of the stretchable wire adjacent to the plurality of electrode elements.

2. The surface electrode according to claim 1, wherein, in a plan view of a cross section orthogonal to the first surface, the stretchable wire has a contour containing a straight line parallel to the first surface and a curve.

3. The surface electrode according to claim 2, wherein the surface electrode further has a second surface opposite the first surface, and
a distance between the straight line and the first surface is shorter than a distance between the straight line and the second surface.

4. The surface electrode according to claim 2, wherein the surface electrode further has a second surface opposite the first surface, and
a distance between the straight line and the first surface is longer than a distance between the straight line and the second surface.

5. The surface electrode according to claim 2, wherein a total length of the curve is longer than a total length of the straight line.

6. The surface electrode according to claim 1, wherein, in a plan view of the first surface viewed in a direction orthogonal to the first surface, an area occupied by the plurality of electrode elements is greater than an area outside the electrode elements.

7. The surface electrode according to claim 1, wherein a shortest distance between two adjacent electrode elements of the plurality of electrode elements is greater than a thickness of any of the plurality of electrode elements.

8. The surface electrode according to claim 1, wherein the surface electrode further has a second surface opposite the first surface,
the surface electrode further comprising a substrate on the second surface, the substrate being made of a material that is softer than that of the plurality of electrode elements.

9. The surface electrode according to claim 8, wherein the material of the substrate is harder than that of the stretchable wire.

10. The surface electrode according to claim 8, wherein the material of the substrate is softer than that of the stretchable wire.

11. The surface electrode according to claim 1, further comprising a via between the stretchable wire and the plurality of electrode elements, the via electrically connecting the stretchable wire to the plurality of electrode elements,
wherein in a mixture of a conductive material and a resin material forming the via, the conductive material contains a carbon-based conductive material.

12. The surface electrode according to claim 1, further comprising a via between the stretchable wire and the plurality of electrode elements, the via being configured to electrically connect the stretchable wire to the plurality of electrode elements,
wherein in plan view of a cross section orthogonal to the first surface, the stretchable wire extends beyond an outside diameter of an end portion of the via toward an end portion of the surface electrode, as viewed in a direction orthogonal to the cross section.

13. The surface electrode according to claim 1, wherein a distance between the stretchable wire and a second surface opposite the first surface is shorter than a distance between the stretchable wire and the first surface.

14. The surface electrode according to claim 1, wherein a length of the stretchable wire in a direction toward a closest electrode element of the plurality of electrode elements on the first surface is longer than a length of the closest electrode element of the plurality of electrode elements.

15. The surface electrode according to claim 1, wherein a first distance between a first set of adjacent electrode elements of the plurality of electrode elements spaced apart in an expansion and contraction direction of the measured surface is longer than a second distance between a second set of adjacent electrode elements of the plurality of electrode elements spaced apart in a direction perpendicular to the expansion and contraction direction.

16. The surface electrode according to claim 1, wherein the plurality of electrode elements each have a protrusion protruding in a direction orthogonal to the first surface, and the protrusion is embedded in the stretchable insulator or in the stretchable wire.

17. The surface electrode according to claim 1, further comprising a magnet,
wherein the stretchable wire contains a liquid and a ferromagnetic material; and
in plan view of the magnet viewed in a direction normal to the first surface, the magnet overlaps at least one of the electrode elements of the plurality of electrode elements.

18. The surface electrode according to claim 1, further comprising a sealing portion sealing the stretchable wire,
wherein the sealing portion includes a first sealing portion and a second sealing portion disposed outside the first sealing portion.

19. The surface electrode according to claim 18, wherein a modulus of elasticity of the first sealing portion is greater than a modulus of elasticity of the second sealing portion.

20. The surface electrode according to claim 18, wherein a modulus of elasticity of the second sealing portion is greater than a modulus of elasticity of the first sealing portion.

21. The surface electrode according to claim 18, wherein the stretchable wire contains a liquid; and
the first sealing portion contains a porous material.

22. The surface electrode according to claim 1, wherein the stretchable wire contains a liquid and a porous material.
